(19) United States
(12) Patent Application Publication
(10) Pub. No.: US 2024/0000783 A1
(43) Pub. Date: Jan. 4, 2024

(54) COMBINATION THERAPY

(71) Applicants: Pfizer Inc., New York, NY (US); Astellas Pharma Inc., Tokyo (JP)

(72) Inventors: Lars ANDERS, San Diego, CA (US); Kimberly Hyunjung KIM, San Diego, CA (US); Danan LI, San Diego, CA (US); Elizabeth Anne MCMILLAN, Vista, CA (US); Robert Andrew ROLLINS, New York, NY (US); Brendan Ayers VEENEMAN, Stoneham, MA (US)

(73) Assignees: Pfizer Inc., New York, NY (US); Astellas Pharma Inc., Tokyo (JP)

(21) Appl. No.: 18/041,245

(22) PCT Filed: Aug. 10, 2021
(86) PCT No.: PCT/IB2021/057379

§ 371 Date: Feb. 10, 2023

Related U.S. Application Data
(60) Provisional application No. 63/065,160, filed on Aug. 13, 2020.

(51) Int. Cl. A61K 31/506; A61K 31/4166; A61K 45/06; A61P 35/00

(52) U.S. Cl. CPC A61K 31/506; A61K 31/4166; A61K 45/06; A61P 35/00

(57) ABSTRACT

This invention relates to combination therapies comprising a cyclin dependent kinase 4 (CDK4) inhibitor of Formula (I) or a pharmaceutically acceptable salt thereof, and an antiandrogen, optionally in further combination with an additional anti-cancer agent, and associated methods of treatment, pharmaceutical compositions, and uses thereof.

### BACKGROUND OF THE INVENTION

### Field of the Invention

The present invention relates to combination therapies useful for treating cancer. In particular, the invention relates to combination therapies comprising a cyclin dependent kinase 4 (CDK4) inhibitor of Formula (I) or a pharmaceutically acceptable salt thereof, and an antiandrogen, optionally in further combination with an additional anti-cancer agent. The invention also relates to associated methods of treatment, pharmaceutical compositions, and pharmaceutical uses.

### Description of the Related Art

The androgen receptor (AR) is an androgen-stimulated transcription factor that is known to play a role in promoting certain cancers, including the development and progression of prostate cancer, certain breast cancers, certain lung cancers, hepatocellular carcinoma, and salivary gland tumors, among other cancers. Testosterone and other male sex hormones, known collectively as androgens, can fuel the growth of prostate cancer cells by binding to and activating the androgen receptor. Initial treatment for advanced prostate cancer may involve reducing the amounts of androgens produced by the body, primarily in the testes. This can be achieved surgically by removal of both testicles (bilateral orchiectomy) or through use of hormone deprivation therapies such as luteinizing hormone-releasing hormone (LHRH) agonist or antagonist drugs, which lower the native production of testosterone (sometimes called “chemical castration”). However, over time resistance is known to develop to these hormone deprivation therapies, leading to an aggressive form of prostate cancer known as castration-resistant prostate cancer (CRPC), or hormone-refractory prostate cancer. This resistance is thought to be related to amplification and/or over-expression of the androgen receptor. Once in this state, prostate cancers generally continue to grow despite the reduction of testosterone production to very low (i.e. post-castration) levels. The progression to castration-resistant prostate cancer may be determined based on either rising levels of prostate-specific antigen (PSA), or documented disease progression as evidenced by imaging tests or clinical symptoms.

Antiandrogens are thought to suppress androgen activity by a number of different mechanisms. One example of an antiandrogen approved for the treatment of castration-resistant prostate cancer is abiraterone acetate (marketed as Zytiga™), a steroidal CY17A1 inhibitor. One specific class of antiandrogens are androgen receptor inhibitors, also known as androgen receptor antagonists, which are thought to compete with endogenous ligands, androgens, for the androgen receptor. When an antagonist binds to an androgen receptor it is thought to induce a conformational change in the receptor itself that impedes transcription of key androgen regulated genes and therefore inhibits the biological effects of the androgens themselves, such as testosterone and dihydrotestosterone. Enzalutamide (marketed as XTANDI®) is a non-steroidal androgen receptor inhibitor approved for the treatment of metastatic castration-resistant prostate cancer. However, despite treatment with antiandrogens, for some subjects, their cancer will relapse or the subjects may develop therapeutic resistance. The mechanisms that underlie such resistance are, to date, not yet fully understood.

Cyclin-dependent kinases (CDKs) and related serine/threonine protein kinases are important cellular enzymes that perform essential functions in regulating cell division and proliferation. CDKs 1-4, 6, 10, 11 have been reported to play a direct role in cell cycle progression, while CDKs 3, 5 and 7-9 may play an indirect role (e.g., through activation of other CDKs, regulation of transcription or neuronal functions). The CDK catalytic units are activated by binding to regulatory subunits, known as cyclins, followed by phosphorylation. Cyclins can be divided into four general classes (G₁, G₁/S, S and M cyclins) whose expression levels vary at different points in the cell cycle. Cyclin B/CDK1, cyclin A/CDK2, cyclin E/CDK2, cyclin D/CDK4, cyclin D/CDK6, and likely other heterodynes are important regulators of cell cycle progression.

Combinatorial targeted therapy has emerged as a promising strategy to intensify single agent anti-tumor activity and to counter acquired cancer drug resistance (Al-Lazikani et al, Combinatorial drug therapy for cancer in the post-genomic era, Nat. Biotechnol. (2012), 30:679-92). Single agent activity of CDK inhibitors in the clinic has been generally disappointing. Hence, prevailing evidence supports clinical approaches where a CDK inhibitor is combined with another anti-cancer agent to maximize anti tumor efficacy of targeted therapy (Dickson and Schwartz, Development of cell-cycle inhibitors for cancer therapy, Curr. Oncol. (2009), 16:36-43).

CDK4/6 inhibitors, including palbociclib, ribociclib and abemaciclib, have been approved for treatment of hormone receptor (HR)-positive, human epidermal growth factor receptor 2 (HER2)-negative (HR+/HER2−) advanced or metastatic breast cancer in combination with endocrine therapy, based on enhanced efficacy in prolonging PFS when compared to patients treated with endocrine therapy alone (Serra et al, Palbociclib in metastatic breast cancer: current evidence and real-life data, Drugs Context. (2019), 8:212579). CDK4/6 inhibition plus estrogen receptor (ER) blockade was shown to elicit additive anti-proliferative effects against HR+/HER2− breast cancer cells in vitro (Finn et al, PD 0332991, a selective cyclin D kinase 4/6 inhibitor, preferentially inhibits proliferation of luminal estrogen receptor-positive human breast cancer cell lines in vitro, Breast Cancer Res. (2009), 11:R77).

The estrogen receptor (ER) positively regulates expression of cyclin D1, the activating subunit of CDK4, thereby driving cell cycle entry (Foster & Wimalasena, Estrogen regulates activity of cyclin-dependent kinases and retinoblastoma protein phosphorylation in breast cancer cells, Mol. Endocrinol. (1996), 10:488-98). Based on this, the prevailing view is that the combinatorial benefit may be attributed, at least in part, to convergent inhibitory effects of CDK4/6 and ER inhibitors on the cyclin D-CDK4/6 complex in breast cancer cells (VanArsdale et al., Molecular Pathways: Targeting the Cyclin D-CDK4/6 Axis for Cancer Treatment, Clin. Cancer Res. (2015), 21:2905-10). While CDK4/6 inhibitors have shown significant clinical efficacy in HR-positive, HER2-negative advance or metastatic breast cancer, as with drugs targeting other kinases, their effects may be limited over time by the development of primary or acquired resistance.

Numerous oncogenes (in addition to ER) can fuel expression of cyclin D1 and activate CDK4, depending on the cellular context (Choi & Anders, Signaling through cyclin D-dependent kinases, Oncogene (2013), 33:1890-903). One prominent example is the androgen receptor (AR) in prostate cancer cells. In androgen receptor (AR)-positive prostate cancer, AR activation leads to increased levels of cyclin D proteins via post-translational mechanisms (Xu et al., Androgens induce prostate cancer cell proliferation through mammalian target of rapamycin activation and post-transcriptional increases in cyclin D proteins, Cancer Res. (2006), 66:7783-92.

The compound 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2, 3-dideoxy-D-threo-pentitol (COMPOUND A) is a potent and selective inhibitor of CDK4, having the structure:

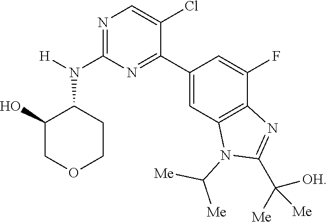 [SMILES: CC(C)n1c(C(C)(C)O)nc2c(F)cc(-c3nc(N[C@@H]4CCOC[C@H]4O)ncc3Cl)cc21]

Compounds of Formula (I), including COMPOUND A, and pharmaceutically acceptable salts thereof are described in International Publication No. WO 2019/207463 and U.S. Publication No. 2019/0330196, the contents of which are incorporated herein by reference in their entirety.

There remains a need for improved therapies for the treatment of cancers. The combinations, methods and uses of the present invention are believed to have one or more advantages, such as greater efficacy than treatment with either therapeutic agent alone; potential to reduce drug-drug interactions; potential to enable an improved dosing schedule; potential to reduce side effects; potential to overcome resistance mechanisms and the like.

### BRIEF SUMMARY OF THE INVENTION

This invention relates to methods, combinations, uses, pharmaceutical compositions and kits for treating abnormal cell growth, particularly cancer, comprising a CDK4 inhibitor of Formula (I) or a pharmaceutically acceptable salt thereof, and an antiandrogen, optionally in further combination with an additional anti-cancer agent.

The invention provides methods, combinations, uses, pharmaceutical compositions and kits, comprising a compound of Formula (I):

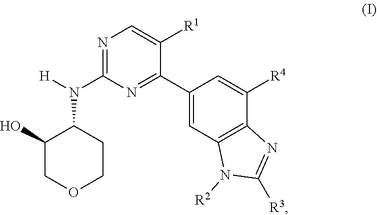

or a pharmaceutically acceptable salt thereof, wherein:
R¹ is H, F or Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy.

In one aspect, the invention provides a method of treating cancer in a subject in need thereof comprising administering to the subject:
(a) an amount of a compound of Formula (I):

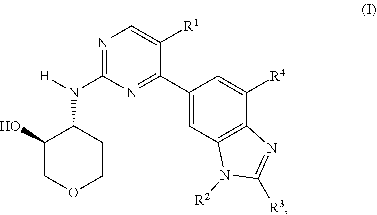

or a pharmaceutically acceptable salt thereof, wherein:
R¹ is H, F or Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy; and
(b) an amount of an antiandrogen;
wherein the amounts in (a) and (b) together are effective in treating cancer.

In some embodiments of this aspect, the invention provides a method further comprising administering to the subject: (c) an amount of an additional anti-cancer agent; wherein the amounts in (a), (b) and (c) together are effective in treating cancer.

In another aspect, the invention provides a combination comprising:
(a) a compound of Formula (I):

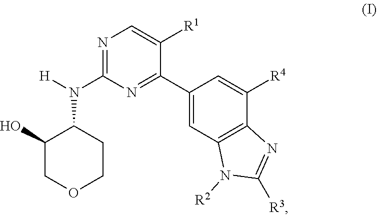

or a pharmaceutically acceptable salt thereof, wherein:
R¹ is H, F or Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy; and
(b) an antiandrogen;
wherein the combination of (a) and (b) is effective in treating cancer.

In some embodiments of this aspect, the combination further comprises (c) an additional anti-cancer agent; wherein the combination of (a), (b) and (c) is effective in treating cancer.

In another aspect, the invention provides a combination for use in treating cancer comprising:
(a) a compound of Formula (I):

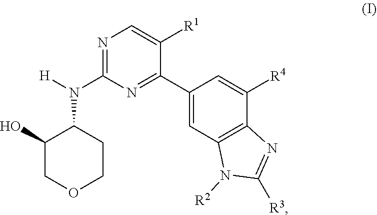

or a pharmaceutically acceptable salt thereof, wherein:
R¹ is H, F or Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy; and
(b) an antiandrogen.

In some embodiments of this aspect, the combination for use further comprises (c) an additional anti-cancer agent.

In another aspect, the invention provides use of a combination comprising:
(a) a compound of Formula (I):

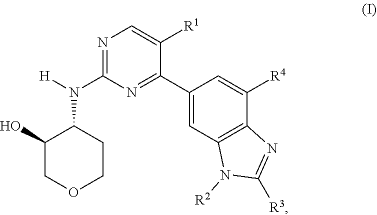

or a pharmaceutically acceptable salt thereof, wherein:
R¹ is H, F or Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy; and
(b) an antiandrogen;
wherein use of the combination is effective in treating cancer.

In some embodiments of this aspect, the combination further comprises (c) an additional anti-cancer agent, wherein the use of the combination of (a), (b) and (c) is effective in treating cancer.

In some embodiments of each of the combinations and uses described herein, the combination of (a) and (b) is synergistic and the invention provides the synergistic combination, or use of the synergistic combination, as described. In some embodiments of the combinations and uses described herein, the combination of (a), (b) and (c) is synergistic and the invention provides the synergistic combination, or use of the synergistic combination, as described.

In some embodiments of each of the methods, combinations and uses described herein, the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A), having the structure:

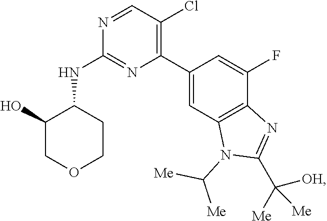 [SMILES: CC(C)n1c(C(C)(C)O)nc2c(F)cc(-c3nc(N[C@@H]4CCOC[C@H]4O)ncc3Cl)cc21]

or a pharmaceutically acceptable salt thereof.

In some embodiments of each of the methods, combinations and uses described herein, the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A).

In some embodiments of each of the methods, combinations and uses described herein, the antiandrogen is selected from the group consisting of enzalutamide, N-desmethyl enzalutamide, darolutamide, apalutamide, and abiraterone, or a pharmaceutically acceptable salt or solvate thereof.

In some embodiments of each of the methods, combinations and uses described herein, the antiandrogen is enzalutamide, or a pharmaceutically acceptable salt or solvate thereof.

In some embodiments of each of the methods, combinations and uses described herein, the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof, and the antiandrogen is selected from the group consisting of enzalutamide, N-desmethyl enzalutamide, darolutamide; apalutamide, and abiraterone, or a pharmaceutically acceptable salt or solvate thereof.

In some embodiments of each of the methods, combinations and uses described herein, the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof, and the antiandrogen is enzalutamide or a pharmaceutically acceptable salt or solvate thereof.

Embodiments of each of the aspects described herein, including embodiments of the methods, combinations and uses of the invention, may be combined with one or more other embodiments of the present invention described herein which is not inconsistent with the embodiment(s) with which it is combined.

### BRIEF DESCRIPTION OF THE DRAWINGS

FIG. 1. Shows dose dependent growth inhibition by COMPOUND A as a single agent (A), and enhanced growth inhibition by the combination of COMPOUND A and enzalutamide over either agent alone (B) in LNCaP human prostate cancer spheroids, as average diameter (μm) at concentrations shown.

FIG. 2. Shows a dose response matrix (A), Loewe excess matrix (B), and isobologram (C) demonstrating the effects of combining COMPOUND A and enzalutamide on proliferation of C₄-3 cells.

FIG. 3. Shows a dose response matrix (A), Loewe excess matrix (B), and isobologram (C) demonstrating the effects of combining COMPOUND A and enzalutamide on proliferation of VCaP cells.

### DETAILED DESCRIPTION OF THE INVENTION

The present invention may be understood more readily by reference to the following detailed description of the preferred embodiments of the invention and the Examples included herein. It is to be understood that the terminology used herein is for the purpose of describing specific embodiments only and is not intended to be limiting. It is further to be understood that unless specifically defined herein, the terminology used herein is to be given its traditional meaning as known in the relevant art.

As used herein, the singular form “a”, “an”, and “the” include plural references unless indicated otherwise. For example, “a” substituent includes one or more substituents.

The invention described herein suitably may be practiced in the absence of any element(s) not specifically disclosed herein. Thus, for example, in each instance herein any of the terms “comprising”, “consisting essentially of”, and “consisting of” may be replaced with either of the other two terms.

“Abnormal cell growth”, as used herein, unless otherwise indicated, refers to cell growth that is independent of normal regulatory mechanisms (e.g., loss of contact inhibition). Abnormal cell growth may be benign (not cancerous), or malignant (cancerous).

The term “about” which used to modify a numerically defined parameter means that the parameter may vary by as much as 10% above or below the stated numerical value for that parameter. For example, a dose of about 5 mg/kg should be understood to mean that the dose may vary between 4.5 mg/kg and 5.5 mg/kg.

The term “administration” and “treatment” as it applies to an animal, human, experimental subject, cell, tissue, organ or biological fluid, refers to contact of an exogenous pharmaceutical, therapeutic agent, diagnostic agent, or pharmaceutical composition, to the animal, human, experimental subject, cell, tissue, organ or biological fluid. Treatment of a cell encompasses contact of a reagent to the cell, as well as contact of a reagent to a fluid, where the fluid is in contact with the cell. “Administration” and “treatment” also means in vitro and ex vivo treatment, e.g., of a cell, by a reagent, diagnostic, binding compound, or by another cell.

As used herein the terms “antiandrogen” and “antiandrogens” refer to compounds that prevent androgens, for example testosterone and dihydrotestosterone (DHT) and the like, from mediating their biological effects in the body. Antiandrogens may act by one or more of the following hormonal mechanisms of action such as blocking and/or inhibiting and/or modulating the androgen receptor (AR); inhibiting androgen production; suppressing androgen production; degrading the AR; inhibiting nuclear translocation; inhibiting binding of the AR to nuclear DNA; and the like. Antiandrogens include, but are not limited to, steroidal androgen receptor inhibitors (for example, cyproterone acetate, spironolactone, megestrol acetate, chlormadinone acetate, oxendolone, and osaterone acetate), non-steroidal androgen receptor inhibitors (for example, enzalutamide, bicalutamide, nilutamide, flutamide, topilutamide), androgen synthesis inhibitors, androgen receptor degraders and the like.

“Angiogenesis” as used herein refers to blood vessel formation. Tumor angiogenesis is the growth of new blood vessels that tumors need to grow. This process is caused by the release of chemicals by the tumor and by host cells near the tumor.

“Apoptosis” as used herein refers to the death of cells that occurs as a normal and controlled part of an organism's growth or development. Apoptosis is a type of cell death in which a series of molecular steps in a cell lead to its death. Apoptosis is one method the body uses to get rid of unneeded or abnormal cells. The process of apoptosis may be blocked in cancer cells.

The terms “cancer”, “cancerous”, or “malignant” refer to or describe the physiological condition in mammals that is typically characterized by unregulated cell growth. As used herein “cancer” refers to any malignant and/or invasive growth or tumor caused by abnormal cell growth. As used herein “cancer” refers to solid tumors named for the type of cells that form them, as well as cancer of blood, bone marrow, or the lymphatic system. Examples of solid tumors include but not limited to sarcomas and carcinomas. Examples of cancers of the blood include but not limited to leukemias, lymphomas and myeloma. The term “cancer” includes but is not limited to a primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type from latter one.

The term “patient” or “subject” refer to any single subject for which therapy is desired or that is participating in a clinical trial, epidemiological study or used as a control, including humans and mammalian veterinary patients such as cattle, horses, dogs and cats. In some embodiments, the subject is a human.

In some embodiments of each of the methods, combinations and uses described herein, the patient or subject: (1) may have histologically or cytologically confirmed adenocarcinoma of the prostate; (2) may have asymptomatic or mildly symptomatic metastatic castration-resistant prostate cancer; (3) may have been surgically or medically castrated, with serum testosterone≤ 50 ng/dL (≤ 1.73 nmol/L) at screening; (4) may be receiving ongoing androgen deprivation therapy (ADT) with a gonadotropin releasing hormone (GnRH) agonist or antagonist for patients who have not undergone bilateral orchiectomy; (5) may have metastatic disease in bone documented on bone scan or in soft tissue documented on CT/MRI scan; (6) may have progressive disease at study entry in the setting of medical or surgical castration as defined by one or more of the following three criteria: (i) prostate specific antigen (PSA) progression defined by a minimum of two rising PSA values from 3 assessments with an interval of at least 7 days between assessments; (ii) soft tissue disease progression as defined by RECIST 1.1; and (iii) bone disease progression defined by Prostate Cancer Working Group 3 (PCWG3) with two or more new metastatic bone lesions on a whole body radionuclide bone scan; and (7) may have an Eastern Cooperative Oncology Group (ECOG) performance status≤ 1. Life expectancy≥12 months as assessed by the investigator.

In some embodiments of each of the methods, combinations and uses described herein, the patient or subject is an adult human. In some embodiments, the subject is a woman of any menopausal status or a man. In some embodiments, the subject is a post-menopausal woman or a man. In some embodiments, the subject is a post menopausal woman. In some embodiments, the subject is a pre-menopausal or peri-menopausal woman. In some embodiments, the subject is a pre-menopausal or peri-menopausal woman treated with a luteinizing hormone-releasing hormone (LHRH) agonist. In some such embodiments, the subject is a man. In some embodiments, the subject is a man treated with an GnRH agonist.

The terms “treat” or “treating” or “treatment” of a cancer as used herein means to administer a combination therapy according to the present invention to a subject having cancer, or diagnosed with cancer, to achieve at least one positive therapeutic effect, such as, for example, reduced number of cancer cells, reduced tumor size, reduced rate of cancer cell infiltration into peripheral organs, or reduced rate of tumor metastases or tumor growth, reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

For the purposes of this invention, beneficial or desired clinical results include, but are not limited to, one or more of the following: reducing the proliferation of (or destroying) neoplastic or cancerous cell; inhibiting metastasis or neoplastic cells; shrinking or decreasing the size of a tumor; remission of the cancer; decreasing symptoms resulting from the cancer; increasing the quality of life of those suffering from the cancer; decreasing the dose of other medications required to treat the cancer; delaying the progression of the cancer; curing the cancer; overcoming one or more resistance mechanisms of the cancer; and/or prolonging survival of patients having the cancer. Positive therapeutic effects in cancer can be measured in a number of ways (see, for example, W. A. Weber, Assessing tumor response to therapy, J. Nucl. Med. 50 Suppl. 1:1S-10S (2009). For example, with respect to tumor growth inhibition (T/C), according to the National Cancer Institute (NCI) standards, a T/C less than or equal to 42% is the minimum level of anti-tumor activity. A T/C<10% is considered a high anti tumor activity level, with T/C (%)=median tumor volume of the treated/median tumor volume of the control×100.

In some embodiments, the treatment achieved by a combination of the invention is any of the partial response (PR), complete response (CR), overall response (OR), objective response rate (ORR), progression free survival (PFS), radiographic PFS, metastasis fee survival (MFS), disease free survival (DFS) and overall survival (OS).

As used herein, the term “complete response” or “CR” means the disappearance of all signs of cancer (e.g., disappearance of all target lesions) in response to treatment. This does not always mean the cancer has been cured.

As used herein, the term “disease-free survival” (DFS) means the length of time after primary treatment for a cancer ends that the patient survives without any signs or symptoms of that cancer.

As used herein, the term “duration of response” (DoR) means the length of time that a tumor continues to respond to treatment without the cancer growing or spreading. Treatments that demonstrate improved DoR can produce a durable, meaningful delay in disease progression.

As used herein, the terms “objective response” and “overall response” refer to a measurable response, including complete response (CR) or partial response (PR). The term “overall response rate” (ORR) refers to the sum of the complete response (CR) rate and the partial response (PR) rate.

As used herein, the term “overall survival” (OS) means the length of time from either the date of diagnosis or the start of treatment for a disease, such as cancer, that patients diagnosed with the disease are still alive. OS is typically measured as the prolongation in life expectancy in patients who receive a certain treatment as compared to patients in a control group (i.e., taking either another drug or a placebo).

As used herein, the term “partial response” or “PR” refers to a decrease in the size of one or more tumors or lesions, or in the extent of cancer in the body, in response to treatment. For example, in some embodiments, PR refers to at least a 30% decrease in the sum of the longest diameters (SLD) of target lesions, taking as reference the baseline SLD.

As used herein, the term “progression free survival” or “PFS” refers to the length of time during and after treatment during which the disease being treated (e.g., cancer) does not get worse. PFS, also referred to as “Time to Tumor Progression”, may include the amount of time patients have experienced a CR or PR, as well as the amount of time patients have experienced SD.

As used herein, the term “progressive disease” or “PD” refers to a cancer that is growing, spreading or getting worse. In some embodiments, PR refers to at least a 20% increase in the SLD of target lesions, taking as reference the smallest SLD recorded since the treatment started, or to the presence of one or more new lesions.

As used herein, the term “stable disease” (SD) refers to a cancer that is neither decreasing nor increasing in extent or severity.

As used herein, the term “sustained response” refers to the sustained effect on reducing tumor growth after cessation of a treatment. For example, the tumor size may be the same size or smaller as compared to the size at the beginning of the medicament administration phase. In some embodiments, the sustained response has a duration of at least the same as the treatment duration, at least 1.5×, 2×, 2.5×, or 3× length of the treatment duration, or longer.

The anti-cancer effect of the method of treating cancer, including “objective response,” “complete response,” “partial response,” “progressive disease,” “stable disease,” “progression free survival,” “duration of response,” as used herein, may be defined and assessed by the investigators using RECIST v1.1 (Eisenhauer et al., New response evaluation criteria in solid tumours: Revised RECIST guideline (version 1.1), Eur J of Cancer, 2009; 45(2):228-47).

In some embodiments of each of the methods, combinations and uses described herein, the therapeutic effect achieved by the compound of Formula (I), e.g., COMPOUND A, in combination with an antiandrogen, and optionally in further combination with an additional anti-cancer agent is defined by reference to any of the following: complete response (CR), disease free survival (DFS), duration of response (DoR), overall response rate (ORR), overall survival (OS), partial response (PR), or progression free survival (PFS). In some embodiments, response to a combination of the invention is any of PR, CR, PFS, DFS, OR OS that is assessed using Response Evaluation Criteria in Solid Tumors (RECIST) 1.1 response criteria.

In some embodiments of each of the methods, combinations and uses described herein, the invention relates to neoadjuvant therapy, adjuvant therapy, first-line therapy, second-line therapy, second-line or later lines of therapy, or third-line or later lines of therapy. In each case as further described herein, the cancer may be localized, advanced or metastatic, and the intervention may occur at point along the disease continuum (i.e., at any stage of the cancer).

In some embodiments of each of the methods, combinations and uses described herein, the treatment achieved by a combination of the invention is measured by the time to PSA progression, the time to initiation of cytotoxic chemotherapy, or the proportion of patients with PSA response greater than or equal to 50%.

The treatment regimen for a method, combination or use of the invention that is effective to treat cancer in a subject may vary according to factors such as the disease state, age, and weight of the subject, and the ability of the therapy to elicit an anti-cancer response in the subject. While an embodiment of any of the aspects of the invention may not be effective in achieving a positive therapeutic effect in every subject, it should do so in a statistically significant number of subjects as determined by any statistical test known in the art such as, but not limited to, the Cox log-rank test, the Cochran-Mantel-Haenszel log-rank test, the Student's t-test, the chi2-test, the U-test according to Mann and Whitney, the Kruskal-Wallis test (H-test), Jonckheere-Terpstrat-test and the Wilcon on-test.

The terms “treatment regimen”, “dosing protocol” and “dosing regimen” may be used interchangeably to refer to the dose and timing of administration of each therapeutic agent in a combination of the invention.

Ameliorating” means a reducing to some extent or improving one or more symptoms upon treatment with a combination described herein, as compared to not administering the combination. “Ameliorating” also includes shortening or reduction in duration of a symptom. that is, reducing to some extent, preferably, eliminating

As used herein, an “effective dosage”, “effective amount” or “therapeutically effective amount” of a compound or pharmaceutical composition is the amount that, when used as indicated (which may be alone if used as a single agent or together with other agents if used in combination) is sufficient to affect one or more beneficial or desired outcomes, including preventing, ameliorating or treating the biochemical, histological or behavioral symptoms of the disease, its complications, and intermediate pathological phenotypes presenting during development of the disease. For prophylactic use, beneficial or desired outcomes may include: eliminating or reducing the risk, lessening the severity, or delaying the onset of the disease. For therapeutic use, beneficial or desired outcomes may include: reducing the incidence or ameliorating one or more symptoms of the disease, reducing the dose of another medication used to treat the disease, enhancing the efficacy or safety of another medication used to treat the disease, or delaying the time to disease progression.

In reference to the treatment of cancer, beneficial or desired outcomes provided by the invention may include: (1) reducing the size of the tumor, (2) inhibiting (that is, slowing to some extent, preferably stopping) tumor metastasis, (3) inhibiting to some extent (that is, slowing to some extent, preferably stopping) tumor growth or tumor invasiveness, (4) reducing the incidence or ameliorating (that is, reducing to some extent, preferably, eliminating) one or more signs or symptoms associated with the cancer, (5) decreasing the dosage of another medication required to treat the cancer, (6) enhancing the efficacy or safety of another medication used to treat the cancer, and/or (7) delaying the time to progression of the cancer.

An effective dosage can be administered in one or more administrations. Combination therapy involves administering each of the component drugs in the combination therapy in an amount sufficient to provide an observable improvement over the baseline clinically observable signs and symptoms of the disorder treated with the combination. The effective amount of a compound or pharmaceutical composition when used as part of a combination therapy may be less than the amount of the compound or pharmaceutical composition if used as a single agent to treat the same disorder.

A “non-standard dosing regimen” refers to a regimen for administering an amount of a substance, agent, compound or pharmaceutical composition, which is different from the amount, dose or schedule typically used for that substance, agent, compound or pharmaceutical composition in a clinical or therapeutic setting. A “non-standard dosing regimen”, includes a “non-standard dose” or a “non-standard dosing schedule.”

A “low dose amount regimen” refers to a dosing regimen where the amount of one or more of the substances, agents, compounds or pharmaceutical compositions in the regimen is dosed at a lower amount or dose than typically used in a clinical or therapeutic setting for that agent, for example when that agent is dosed as a single agent therapy.

The retinoblastoma susceptibility gene (RB1) was the first tumor suppressor gene to be molecularly defined. The retinoblastoma gene product, RB, is frequently mutated or deleted in retinoblastoma and osteosarcoma, and is mutated or deleted with variable frequency in other tumor types, such as prostate cancer (including neuroendocrine prostate carcinoma), breast cancer (including triple negative breast cancer, TNBC), lung cancer (including small cell lung cancer, SCLC, and non-small cell lung cancer, NSCLC), liver cancer, bladder cancer, ovarian cancer, uterine cancer, cervical cancer, stomach cancer, esophageal cancer, head and neck cancer, glioblastoma, and lymphoma. In human cancers, the function of RB may be disrupted through neutralization by a binding protein, (e.g., the human papilloma virus-E7 protein in cervical carcinoma; Ishiji, T, 2000, J Dermatol., 27: 73-86) or deregulation of pathways ultimately responsible for its phosphorylation.

By “RB pathway” it is meant the entire pathway of molecular signaling that includes retinoblastoma protein (RB), and other protein/protein families in the pathway, including but not limited to CDK, E2f, atypical protein kinase C, and Skp2. Inactivation of the RB pathway often results from perturbation of p16INK4a, Cyclin D1, and CDK4.

The terms “RB+,” “RB plus,” “RB-proficient” or “RB-positive” may be used to describe cells expressing detectable amounts of functional RB protein. RB-positive includes wild-type and non-mutated RB protein. A wild-type RB (RB-WT) is generally understood to mean that form of the RB protein which is normally present in a corresponding population and which has the function which is currently assigned to this protein. RB-positive may be cells which contain a functional RB gene. Cells which are RB-positive may also be cells that can encode a detectable RB protein function.

The terms “RB−,” “RB minus,” “RB-deficient” or “RB-negative” describe several types of cell where the function of RB is disrupted, including cells which produce no detectable amounts of functional RB protein. Cells that are RB-negative may be cells which do not contain a functional RB gene. Cells that are RB-negative may also be cells that can encode an RB protein, but in which the protein does not function properly.

In some embodiments of each of the methods, combinations and uses described herein, the cancer is characterized as retinoblastoma wild type (RB-WT). In some embodiments of each of the methods, combinations and uses described herein, the cancer is characterized as RB-positive or RB-proficient. Such RB-positive or RB-proficient cancers contain at least some functional retinoblastoma genes. In some embodiments, such RB-WT, RB-positive or RB-proficient cancers are characterized as RB1-WT, RB1-positive or RB1-proficient cancers.

In some embodiments of each of the methods, combinations and uses described herein, the cancer is characterized as RB-negative or RB-deficient. Such RB-negative or RB-deficient cancers may be characterized by loss of function mutations, which may encode missense mutations (i.e., encode the wrong amino acid) or nonsense mutatons (i.e., encode a stop codon). Alternatively, such RB-negative cancers may be characterized by deletion of all or part of the retinoblastoma gene. In some embodiments, such RB-negative or RB-deficient cancers are characterized as RB1-negative or RB1-deficient.

In other embodiments of each of the methods, combinations and uses described herein, the cancer is characterized as RB-positive, RB-proficient or RB-WT. In some such embodiments, the cancer is further characterized as AR-positive.

In some such embodiments of each of the methods, combinations and uses described herein, the cancer is characterized as RB1-positive, RB1-proficient or RB1-WT. In some such embodiments, the cancer is further characterized as AR-positive.

“Tumor” as it applies to a subject diagnosed with, or suspected of having, a cancer refers to a malignant or potentially malignant neoplasm or tissue mass of any size and includes primary tumors and secondary neoplasms. A solid tumor is an abnormal growth or mass of tissue that usually does not contain cysts or liquid areas. Examples of solid tumors are sarcomas, carcinomas, and lymphomas. Leukaemia's (cancers of the blood) generally do not form solid tumors (National Cancer Institute, Dictionary of Cancer Terms).

“Tumor burden” or “tumor load’, refers to the total amount of tumorous material distributed throughout the body. Tumor burden refers to the total number of cancer cells or the total size of tumor(s), throughout the body, including lymph nodes and bone marrow. Tumor burden can be determined by a variety of methods known in the art, such as, e.g., using callipers, or while in the body using imaging techniques, e.g., ultrasound, bone scan, computed tomography (CT), or magnetic resonance imaging (MRI) scans.

The term “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using callipers, or while in the body using imaging techniques, e.g., bone scan, ultrasound, CR or MRI scans.

The term “additive” is used to mean that the result of the combination of two compounds, components or targeted agents is no greater than the sum of each compound, component or targeted agent individually.

The term “synergy” or “synergistic” are used to mean that the result of the combination of two or more compounds, components or targeted agents is greater than the sum of each compound, component or targeted agent individually. This improvement in the disease, condition or disorder being treated is a “synergistic” effect and combinations providing a synergistic effect may be referred to as synergistic combinations. A “synergistic amount” is an amount of the combination of the two compounds, components or targeted agents that results in a synergistic effect, as “synergistic” is defined herein.

Determining a synergistic interaction between one or two components, the optimum range for the effect and absolute dose ranges of each component for the effect may be definitively measured by administration of the components over different dose ranges, or dose ratios to patients in need of treatment. The observation of synergy in in vitro models or in vivo models can be predictive of the effect in humans and other species to measure a synergistic effect. The results of such studies can also be used to predict effective dose and plasma concentration ratio ranges and the absolute doses and plasma concentrations required in humans and other species such as by the application of pharmacokinetic or pharmacodynamics methods.

A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively. Ma & Motsinger-Reif, Current Method for Quantifying Drug Synergism, Proteom. Bioinform (2019) 1(2):43-48; Tang et al., What is Synergy? The Saariselka Agreement Revisited, Front Pharmacol. (2015) Article 181, 6: 1-5.

### CDK4 Inhibitors

The invention relates to methods, combinations, and uses comprising a CDK4 inhibitor, wherein the CDK4 inhibitor is a compound of Formula (I):

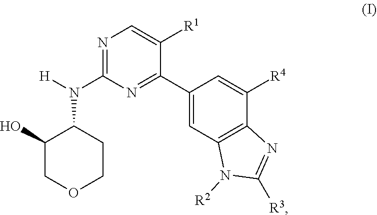

or a pharmaceutically acceptable salt thereof, wherein:
R¹ is H, F or Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy.

In some embodiments, the invention relates to a CDK4 inhibitor of Formula (I), or a pharmaceutically acceptable salt or solvate thereof.

In some embodiments of each of the methods, combinations, and uses described herein, the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A), having the structure:

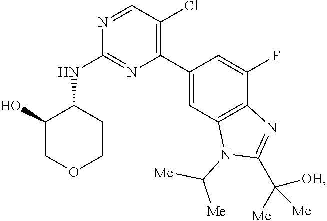 [SMILES: CC(C)n1c(C(C)(C)O)nc2c(F)cc(-c3nc(N[C@@H]4CCOC[C@H]4O)ncc3Cl)cc21]

or a pharmaceutically acceptable salt thereof.

COMPOUND A was prepared as described in Example A94 of U.S. Publication No. 2019/0330196, the contents of which are incorporated herein by reference in their entirety.

In some embodiments of each of the methods, combinations, and uses described herein, the compound of Formula (I) is COMPOUND A, or a pharmaceutically acceptable salt or solvate thereof.

The preparation of compounds of Formula (I), including COMPOUND A, are described in International Application PCT/IB2019/053314, published as WO 2019/207463 on 31 Oct. 2019, and in U.S. application Ser. No. 16/391,836, published as U.S. Publication No. 2019/0330196 on 31 Oct. 2019, the contents of which are incorporated herein by reference in their entirety.

### Antiandrogens

The invention relates to methods, combinations, and uses comprising an antiandrogen, or a pharmaceutically acceptable salt or solvate thereof. In some embodiments, the invention relates to an antiandrogen, or a pharmaceutically acceptable salt thereof.

In some such embodiments, the antiandrogen is a compound which degrades the androgen receptor. In other such embodiments, the antiandrogen is a compound which inhibits or suppresses the production of androgens.

In some embodiments of each of the methods, combinations and uses described herein, the antiandrogen is abiraterone, or a pharmaceutically acceptable salt or solvate thereof, such as abiraterone acetate (marketed as Zytiga™), a steroidal CY17A1 inhibitor which is disclosed in U.S. Pat. No. 5,604,213 which published on 18 Feb. 1997, the contents of which are incorporated herein by reference.

In other embodiments of each of the methods, combinations and uses described herein, the antiandrogen is an androgen receptor inhibitor, or a pharmaceutically acceptable salt or solvate thereof. In some such embodiments, the antiandrogen is an androgen receptor inhibitor, or a pharmaceutically acceptable salt thereof.

Androgen receptor inhibitors useful for the invention include, but are not limited to, non-steroidal small molecule androgen-receptor inhibitors, or pharmaceutically acceptable salts and solvates thereof. Androgen receptor inhibitors can be identified by methods known to those skilled in the art, for example using in vitro assays, cellular ligand binding assays, or gene expression assays such as those disclosed in Tran et al., Development of a second-generation antiandrogen for treatment of advanced prostate cancer, Science, (2009), 324:787-790.

In some embodiments of each of the methods, combinations and uses described herein, the androgen receptor inhibitor is enzalutamide, having the structure:

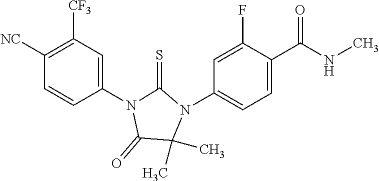 [SMILES: CNC(=O)c1ccc(N2C(=S)N(c3ccc(C#N)c(C(F)(F)F)c3)C(=O)C2(C)C)cc1F]

or a pharmaceutically acceptable salt or solvate thereof.

In some such embodiments, the androgen receptors inhibitor is enzalutamide, or a pharmaceutically acceptable salt thereof. In some such embodiments, the androgen receptor inhibitor is enzalutamide. Enzalutamide is also known as RD162′; 4-[3-[4-cyano-3-(trifluoromethyl)phenyl]-5,5-dimethyl-4-oxo-2-thioxo-1-imidazolidinyl]-2-fluoro-N-methyl-benzamide; or 4-{3-[4-cyano-3-(trifluoromethyl)-phenyl]-5,5-dimethyl-4-oxo-2-sulfanylideneimidazolidin-1-yl}-2-fluoro-N-methylbenzamide; which is disclosed in PCT/US2006/011417, which published on 23 Nov. 2006 as WO 2006/124118, the contents of which are included herein by reference.

In some embodiments of each of the methods, combinations and uses described herein, the androgen receptor inhibitor is N-desmethyl enzalutamide, having the structure:

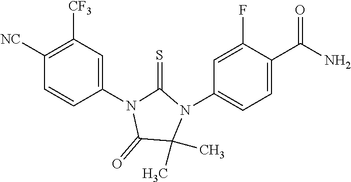 [SMILES: CC1(C)C(=O)N(c2ccc(C#N)c(C(F)(F)F)c2)C(=S)N1c1ccc(C(N)=O)c(F)c1]

or a pharmaceutically acceptable salt or solvate thereof.

N-desmethyl enzalutamide is also known as 4-[3-[4-cyano-3-(trifluoromethyl)phenyl]-5,5-dimethyl-4-oxo-2-thioxoimidazolidin-1-yl]-2-fluorobenzamide; or MII; which is disclosed in PCT/US2010/025283, which published on 2 Sep. 2010 as WO 2010/099238, the contents of which are included herein by reference.

In some embodiments of each of the methods, combinations and uses described herein, the androgen receptor inhibitor is apalutamide, having the structure:

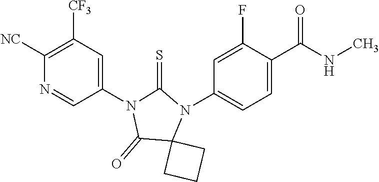 [SMILES: CNC(=O)c1ccc(N2C(=S)N(c3cnc(C#N)c(C(F)(F)F)c3)C(=O)C23CCC3)cc1F]

or a pharmaceutically acceptable salt or solvate thereof.

Apalutamide is also known as ARN-509; or 4-{7-[6-cyano-5-(trifluoromethyl)pyridine-3-yl]-8-oxo-6-thioxo-5,7-diazaspiro[3,4]octan-5yl}-2-fluoro-N-methylbenzamide; which is disclosed in PCT/US2007/007485, which published on 8 Nov. 2007 as WO 2007/126765, the contents of which are included herein by reference. In one embodiment, the androgen receptor inhibitor useful in the present invention is a pharmacologically active metabolite of apalutamide, or a pharmaceutically acceptable salt or solvate thereof.

In some embodiments of each of the methods, combinations and uses described herein, the androgen receptor inhibitor is HC-1119, having the structure:

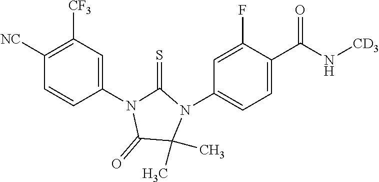 [SMILES: [2H]C([2H])([2H])NC(=O)c1ccc(N2C(=S)N(c3ccc(C#N)c(C(F)(F)F)c3)C(=O)C2(C)C)cc1F]

or a pharmaceutically acceptable salt or solvate thereof.

HC-1119 is disclosed in PCT/CN2012/086573, which published on 20 Jun. 2013 as WO 2013/087004, and U.S. Pat. No. 9,346,764, the contents of each of which are included herein by reference.

In some embodiments of each of the methods, combinations and uses described herein, the androgen receptor inhibitor is ONO1-00136, having the structure:

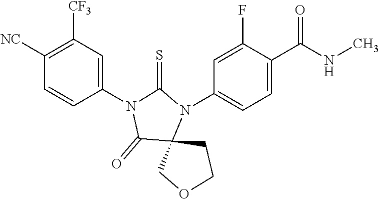 [SMILES: CNC(=O)c1ccc(N2C(=S)N(c3ccc(C#N)c(C(F)(F)F)c3)C(=O)[C@]23CCOC3)cc1F]

or a pharmaceutically acceptable salt or solvate thereof.

ONO1-00136 is disclosed in PCT/RU2011/000476, which published on 26 Jan. 2012 as WO 2012/011840, and RU 2434851, the contents of each of which are included herein by reference.

In some embodiments of each of the methods, combinations and uses described herein, the androgen receptor inhibitor is darolutamide, having the structure:

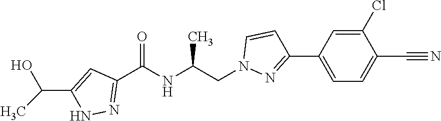 [SMILES: CC(O)c1cc(C(=O)N[C@@H](C)Cn2ccc(-c3ccc(C#N)c(Cl)c3)n2)n[nH]1]

or a pharmaceutically acceptable salt or solvate thereof.

Darolutamide is also known as N-[(2S)-1-[3-(3-chloro-4-cyanophenyl)-1H-pyrazol-1-yl]propan-2-yl]-5-(1-hydroxyethyl)-1H-pyrazole-3-carboxamide which is disclosed in PCT/FI2010/000065, which published on 5 May 2011 as WO 2011/051540, the contents of which are included herein by reference.

In some embodiments of each of the methods, combinations and uses described herein, the androgen receptor inhibitor is bicalutamide, having the structure:

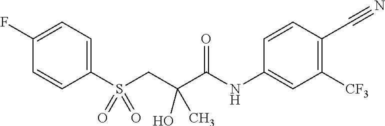 [SMILES: CC(O)(CS(=O)(=O)c1ccc(F)cc1)C(=O)Nc1ccc(C#N)c(C(F)(F)F)c1]

or a pharmaceutically acceptable salt or solvate thereof.

Bicalutamide is marketed as Casodex™, which is disclosed in U.S. Pat. No. 4,636,505, published on 13 Jan. 1987, the contents of which are included herein by reference.

In some embodiments of each of the methods, combinations and uses described herein, the androgen receptor inhibitor is nilutamide, having the structure:

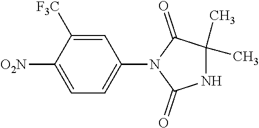 [SMILES: CC1(C)NC(=O)N(c2ccc([N+](=O)[O-])c(C(F)(F)F)c2)C1=O]

or a pharmaceutically acceptable salt or solvate thereof.

In some embodiments of each of the methods, combinations and uses described herein, the androgen receptor inhibitor is flutamide, having the structure:

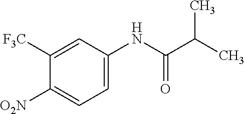 [SMILES: CC(C)C(=O)Nc1ccc([N+](=O)[O-])c(C(F)(F)F)c1]

or a pharmaceutically acceptable salt or solvate thereof.

In some embodiments of each of the methods, combinations and uses described herein, the antiandrogen is selected from the group consisting of enzalutamide, N desmethyl enzalutamide, darolutamide, apalutamide, and abiraterone, or a pharmaceutically acceptable salt or solvate thereof.

In some embodiments of each of the methods, combinations and uses described herein, the antiandrogen is an androgen receptor inhibitor, wherein the androgen receptor inhibitor is selected from the group consisting of enzalutamide, N-desmethyl enzalutamide, darolutamide, apalutamide, and abiraterone, or a pharmaceutically acceptable salt or solvate thereof.

In some embodiments of each of the methods, combinations and uses described herein, the antiandrogen is administered in further combination with androgen deprivation therapy (ADT). In some such embodiments, the ADT is selected from the group consisting of a luteinizing hormone-releasing hormone (LHRH) agonist, a LHRH antagonist, a gonadotropin releasing hormone (GnRH) agonist and a GnRH antagonist. In some such embodiments, the ADT is selected from the group consisting of leuprolide (also known as leuprorelin, for example Lupron or Eligardor Viadur and the like); buserelin (for example Suprefact); gonadorelin; goserelin (for example Zoladex); histrelin (for example Vantas); nafarelin; triptorelin (for example Trelstar); deslorelin; fertirelin; abarelix (for example Plenaxis); cetrorelix; degarelix (for example Firmagon); ganirelix; ozarelix; elagolix (for example Orilissa); relugolix; and linzagolix.

In some such embodiments, the ADT is leuprolide. In some such embodiments, the ADT is goserelin. In other such embodiments, the ADT is degarelix.

In some embodiments of each of the methods, combinations and uses described herein, the antiandrogen is enzalutamide administered in further combination with ADT, wherein the ADT is selected from the group consisting of leuprolide, buserelin, gonadorelin, goserelin, histrelin, nafarelin, triptorelin, deslorelin, fertirelin, abarelix, cetrorelix, degarelix, ganirelix, ozarelix, elagolix, relugolix and linzagolix, or a pharmaceutically acceptable salt thereof. In some such embodiments, the antiandrogen is enzalutamide administered in further combination with ADT, wherein the ADT is selected from the group consisting of leuprolide, goserelin and degarelix.

In some embodiments of each of the methods, combinations and uses described herein, the antiandrogen is N-desmethyl enzalutamide administered in further combination with ADT, wherein the ADT is selected from the group consisting of leuprolide, buserelin, gonadorelin, goserelin, histrelin, nafarelin, triptorelin, deslorelin, fertirelin, abarelix, cetrorelix, degarelix, ganirelix, ozarelix, elagolix, relugolix and linzagolix, or a pharmaceutically acceptable salt thereof. In some such embodiments, the antiandrogen is N-desmethyl enzalutamide administered in further combination with ADT, wherein the ADT is selected from the group consisting of leuprolide, goserelin and degarelix.

In some embodiments of each of the methods, combinations and uses described herein, the antiandrogen is apalutamide administered in further combination with ADT, wherein the ADT is selected from the group consisting of leuprolide, buserelin, gonadorelin, goserelin, histrelin, nafarelin, triptorelin, deslorelin, fertirelin, abarelix, cetrorelix, degarelix, ganirelix, ozarelix, elagolix, relugolix and linzagolix, or a pharmaceutically acceptable salt thereof. In some such embodiments, the antiandrogen is apalutamide administered in further combination with ADT, wherein the ADT is selected from the group consisting of leuprolide, goserelin and degarelix.

In some embodiments of each of the methods, combinations and uses described herein, the antiandrogen is abiraterone, preferably abiraterone acetate, administered in further combination with ADT, wherein the ADT is selected from the group consisting of leuprolide, buserelin, gonadorelin, goserelin, histrelin, nafarelin, triptorelin, deslorelin, fertirelin, abarelix, cetrorelix, degarelix, ganirelix, ozarelix, elagolix, relugolix and linzagolix, or a pharmaceutically acceptable salt thereof. In some such embodiments, the antiandrogen is abiraterone, preferably abiraterone acetate, administered in further combination with ADT, wherein the ADT is selected from the group consisting of leuprolide, goserelin and degarelix.

### Pharmaceutically Acceptable Salts

As used herein, the term “pharmaceutically acceptable salt” refers to those salts which retain the biological effectiveness and properties of the parent compound. The phrase “pharmaceutically acceptable salt(s)”, as used herein, unless otherwise indicated, includes salts of acidic or basic groups which may be present in the compounds of the formulae disclosed herein. For example, the compounds of the invention that are basic in nature may be capable of forming a wide variety of salts with various inorganic and organic acids. The acids that may be used to prepare pharmaceutically acceptable acid addition salts of such basic compounds of those that form non-toxic acid addition salts, i.e., salts containing pharmacologically acceptable anions. Examples of anions suitable for mono- and di-acid addition salts include, but are not limited to, acetate, asparatate, benzenesulfonate, benzoate, besylate, bicarbonate, bisulfate, bitartrate, bromide, calcium edetate, camsylate, carbonate, chloride, citrate, decanoate, edetate, edislyate, estolate, esylate, fumarate, gluceptate, gluconate, glutamate, glycollate, hexanoate, hexylresorcinate, hydrabamine, hydroxynaphthoate, iodide, isethionate, lactate, lactobionate, malate, maleate, mandelate, mesylate, methylsulfate, mucate, napsylate, nitrate, octanoate, oleate, pamoate (embonate), pantothenate, phosphate, polygalacturonate, propionate, salicylate, stearate, subacetate, succinate, sulfate, tannate, tartrate, teoclate, tosylate, triethiodode, and valerate salts. Alternatively, compounds that are acidic in nature may be capable of forming base salts with various pharmacologically acceptable cations which form non-toxic base salts. Such non-toxic base salts include, but are not limited to, those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines. Examples of cations suitable for such salts include alkali metal or alkaline-earth metal salts and other cations, including aluminium, arginine, benzathine, calcium, chloroprocaine, choline, diethanolamine, ethanolamine, ethylenediamine, lysine, magnesium, histidine, lithium, meglumine, potassium, procaine, sodium, triethyamine and zinc. Salts may be prepared by conventional techniques. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002). Methods for making pharmaceutically acceptable salts are known to those of skill in the art.

Unless indicated otherwise, all references herein to CDK4 inhibitors, to compounds of Formula (I), and to antiandrogens include references to pharmaceutically acceptable salts, solvates, hydrates and complexes thereof, and to solvates, hydrates and complexes of pharmaceutically acceptable salts thereof, and include amorphous and polymorphic forms, stereoisomers, and isotopically labeled versions thereof.

### Therapeutic Methods, Combinations, Uses

The present invention provides methods, combinations and uses for treating cancer. Some embodiments provided herein result in one or more of the following effects: (1) inhibiting cancer cell proliferation; (2) inhibiting cancer cell invasiveness; (3) inducing apoptosis of cancer cells; (4) inhibiting cancer cell metastasis; (5) inhibiting angiogenesis; or (6) overcoming one or more resistance mechanisms relating to a cancer treatment.

The present invention provides methods, combinations and uses comprising a compound of Formula (I):

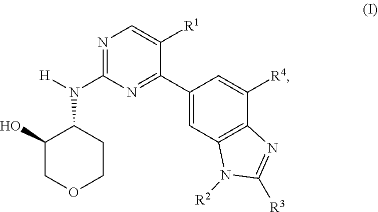

or a pharmaceutically acceptable salt thereof, wherein:
R¹ is H, F or Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy.

In each instance recited herein, reference to “a compound of Formula (I)” may be replaced by “a CDK4 inhibitor of Formula (I).”

In one aspect, the invention provides a method of treating cancer in a subject in need thereof comprising administering to the subject:
(a) an amount of a compound of Formula (I):

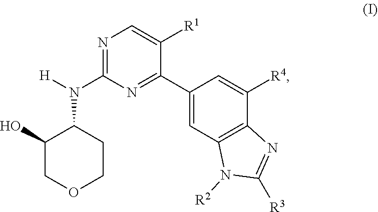

or a pharmaceutically acceptable salt thereof, wherein:
R¹ is H, F or Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy; and
(b) an amount of an antiandrogen;
wherein the amounts in (a) and (b) together are effective in treating cancer.

In some embodiments of this aspect, the invention provides a method further comprising administering to the subject: (c) an amount of an additional anti-cancer agent; wherein the amounts in (a), (b) and (c) together are effective in treating cancer.

In another aspect, the invention provides a combination comprising:
(a) a compound of Formula (I):

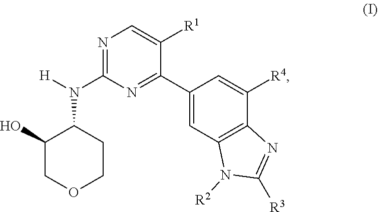

or a pharmaceutically acceptable salt thereof, wherein:
R¹ is H, F or Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy; and
(b) an antiandrogen;
wherein the combination of (a) and (b) is effective in treating cancer.

In some embodiments of this aspect, the combination further comprises (c) an additional anti-cancer agent; wherein the combination of (a), (b) and (c) is effective in treating cancer.

In another aspect, the invention provides a combination for use in treating cancer comprising:
(a) a compound of Formula (I):

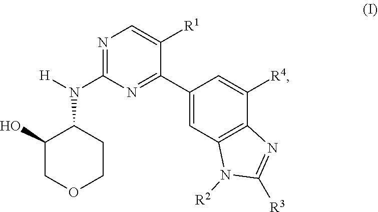

or a pharmaceutically acceptable salt thereof, wherein:
R¹ is H, F or Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy; and
(b) an antiandrogen.

In some embodiments of this aspect, the combination for use further comprises (c) an additional anti-cancer agent.

In another aspect, the invention provides use of a combination comprising:
(a) a compound of Formula (I):

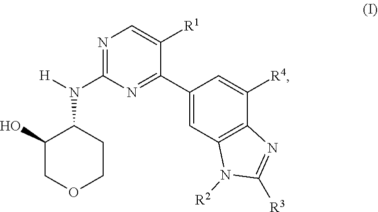

or a pharmaceutically acceptable salt thereof, wherein:
R¹ is H, F or Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy; and
(b) an antiandrogen;
wherein use of the combination of (a) and (b) is effective in treating cancer.

In some embodiments of this aspect, the combination further comprises (c) an additional anti-cancer agent, wherein the use of the combination of (a), (b) and (c) is effective in treating cancer.

In some embodiments of each of the methods, combinations and uses described herein, the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A), having the structure:

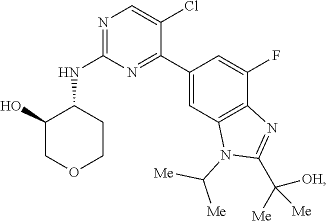 [SMILES: CC(C)n1c(C(C)(C)O)nc2c(F)cc(-c3nc(N[C@@H]4CCOC[C@H]4O)ncc3Cl)cc21]

or a pharmaceutically acceptable salt thereof.

In some embodiments of each of the methods, combinations and uses described herein, the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A), having the structure:

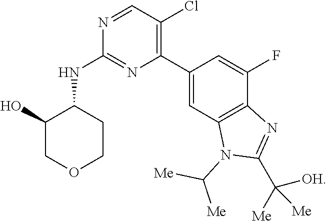 [SMILES: CC(C)n1c(C(C)(C)O)nc2c(F)cc(-c3nc(N[C@@H]4CCOC[C@H]4O)ncc3Cl)cc21]

In another aspect, the invention provides a method of treating cancer in a subject in need thereof comprising administering to the subject:
(a) an amount of 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2, 3-dideoxy-D-threo-pentitol (COMPOUND A) or a pharmaceutically acceptable salt thereof; and
(b) an amount of an antiandrogen;
wherein the amounts in (a) and (b) together are effective in treating cancer.

In another aspect, the invention provides a method of treating cancer in a subject in need thereof comprising administering to the subject:
(a) an amount of 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2, 3-dideoxy-D-threo-pentitol (COMPOUND A) or a pharmaceutically acceptable salt thereof;
(b) an amount of an antiandrogen; and
(c) an amount of an additional anti-cancer agent;
wherein the amounts in (a), (b) and (c) together are effective in treating cancer.

In a preferred aspect, the invention provides a method of treating cancer in a subject in need thereof comprising administering to the subject:
(a) an amount of an amount of 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof; and
(b) an amount of enzalutamide or a pharmaceutically acceptable salt or solvate thereof;
wherein the amounts in (a) and (b) together are effective in treating cancer.

In another preferred aspect, the invention provides a method of treating cancer in a subject in need thereof comprising administering to the subject:
(a) an amount of 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2, 3-dideoxy-D-threo-pentitol (COMPOUND A) or a pharmaceutically acceptable salt thereof;
(b) an amount of enzalutamide or a pharmaceutically acceptable salt or solvate thereof; and
(c) an amount of an additional anti-cancer agent;
wherein the amounts in (a), (b) and (c) together are effective in treating cancer.

In another aspect, the invention provides a combination comprising:
(a) 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof; and
(b) an antiandrogen;
   wherein the combination of (a) and (b) is effective in treating cancer.

In another aspect, the invention provides a combination comprising:
(a) 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2, 3-dideoxy-D-threo-pentitol (COMPOUND A) or a pharmaceutically acceptable salt thereof;
(b) an antiandrogen; and
(c) an additional anti-cancer agent;
wherein the combination of (a), (b) and (c) is effective in treating cancer.

In a preferred aspect, the invention provides a combination comprising:
(a) 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof; and
(b) enzalutamide or a pharmaceutically acceptable salt or solvate thereof;
   wherein the combination of (a) and (b) is effective in treating cancer.

In another preferred aspect, the invention provides a combination comprising:
(a) 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2, 3-dideoxy-D-threo-pentitol (COMPOUND A) or a pharmaceutically acceptable salt thereof;
(b) enzalutamide or a pharmaceutically acceptable salt or solvate thereof; and
(c) an additional anti-cancer agent;
   wherein the combination of (a), (b) and (c) is effective in treating cancer.

In another aspect, the invention provides a combination for use in treating cancer comprising:
(a) 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof; and
(b) an antiandrogen.

In another aspect, the invention provides a combination for use in treating cancer comprising:
(a) 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof;
(b) an antiandrogen; and
(c) an additional anti-cancer agent.

In a preferred aspect, the invention provides a combination for use in treating cancer comprising:
(a) 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof; and
(b) enzalutamide or a pharmaceutically acceptable salt or solvate thereof.

In another preferred aspect, the invention provides a combination for use in treating cancer comprising:
(a) 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof;
(b) enzalutamide or a pharmaceutically acceptable salt or solvate thereof; and
(c) an additional anti-cancer agent.

In another aspect, the invention provides use of a combination comprising:
(a) 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof; and
(b) an antiandrogen;
wherein use of the combination of (a) and (b) is effective in treating cancer.

In another aspect, the invention provides use of a combination comprising:
(a) 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof;
(b) an antiandrogen; and
(c) an additional anti-cancer agent;
wherein use of the combination of (a), (b) and (c) is effective in treating cancer.

In a preferred aspect, the invention provides use of a combination comprising:
(a) 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof; and
(b) enzalutamide or a pharmaceutically acceptable salt or solvate thereof;
wherein use of the combination of (a) and (b) is effective in treating cancer.

In another preferred aspect, the invention provides use of a combination comprising:
(a) 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof;
(b) enzalutamide or a pharmaceutically acceptable salt or solvate thereof; and
(c) an additional anti-cancer agent.
wherein use of the combination of (a), (b) and (c) is effective in treating cancer.

In some embodiments of each of the combinations herein, the combination of (a) and (b) is synergistic and the invention provides the synergistic combination. In some embodiments of the combinations herein, the combination of (a), (b) and (c) is synergistic and the invention provides the synergistic combination.

In some embodiments of each of the combinations for use herein, the combination of (a) and (b) is synergistic and the invention provides the synergistic combination for use in treating cancer as described. In some embodiments of the combinations for use herein, the combination of (a), (b) and (c) is synergistic and the invention provides the synergistic combination for use in treating cancer as described.

In some embodiments of each of the uses described herein, the combination of (a) and (b) is synergistic and the invention provides use of the synergistic combination as described. In some embodiments of the uses described herein, the combination of (a), (b) and (c) is synergistic and the invention provides use of the synergistic combination as described.

In some embodiments of each of the methods, combinations and uses described herein, the compound of Formula (I) or a pharmaceutically acceptable salt thereof, and the antiandrogen or a pharmaceutically acceptable salt or solvate thereof, are administered sequentially, simultaneously or concurrently.

In some embodiments of the methods, combinations and uses described herein, the compound of Formula (I) or a pharmaceutically acceptable salt thereof, the antiandrogen or a pharmaceutically acceptable salt or solvate thereof, and the additional anti-cancer agent are administered sequentially, simultaneously or concurrently.

In some embodiments of each of the methods, combinations and uses described herein, COMPOUND A or a pharmaceutically acceptable salt thereof, and the antiandrogen or a pharmaceutically acceptable salt or solvate thereof, are administered sequentially, simultaneously or concurrently.

In some embodiments of the methods, combinations and uses described herein, COMPOUND A or a pharmaceutically acceptable salt thereof, the antiandrogen or a pharmaceutically acceptable salt or solvate thereof, and the additional anti-cancer agent are administered sequentially, simultaneously or concurrently.

In some embodiments of each of the methods, combinations and uses described herein, COMPOUND A or a pharmaceutically acceptable salt thereof, and enzalutamide or a pharmaceutically acceptable salt or solvate thereof, are administered sequentially, simultaneously or concurrently.

In some embodiments of the methods, combinations and uses described herein, COMPOUND A or a pharmaceutically acceptable salt thereof, enzalutamide or a pharmaceutically acceptable salt or solvate thereof, and the additional anti-cancer agent are administered sequentially, simultaneously or concurrently.

In some embodiments of the methods, combinations and uses described herein, the additional anti-cancer agent is an ADT, wherein the ADT is selected from the group consisting of leuprolide, goserelin and degarelix.

In some embodiments of the methods, combinations and uses described herein, the cancer is selected from the group consisting of prostate cancer, breast cancer, lung cancer (including non-small cell lung cancer, NSCLC, and small cell lung cancer, SCLC), liver cancer (including hepatocellular carcinoma, HCC), kidney cancer (including renal cell carcinoma, RCC), bladder cancer (including urothelial carcinomas, such as upper urinary tract urothelial carcinoma, UUTUC), ovarian cancer (including epithelial ovarian cancer, EOC), peritoneal cancer (including primary peritoneal cancer, PPC), fallopian tube cancer, cervical cancer, uterine cancer (including endometrial cancer), pancreatic cancer, stomach cancer, colorectal cancer, esophageal cancer, head and neck cancer (including squamous cell carcinoma of the head and neck (SCCHN), thyroid cancer, and salivary gland cancer), testicular cancer, adrenal cancer, skin cancer (including basal cell carcinoma and melanoma), brain cancer (including astrocytoma, meningioma, and glioblastoma), sarcoma (including osteosarcoma and liposarcoma), and lymphoma (including mantle cell lymphoma, MCL).

In some embodiments of each of the methods, combinations and uses described herein, the cancer is androgen-dependent.

In some embodiments of each of the methods, combinations and uses described herein, the cancer expresses androgen receptors, which may sometimes be referred to as androgen receptor (AR)-positive or AR+ cancer.

In some embodiments of the methods, combinations and uses described herein, the cancer is advanced or metastatic cancer. In some embodiments of the methods, combinations and uses described herein, the cancer is early stage or non-metastatic cancer.

In some embodiments of the methods, combinations and uses described herein, the cancer is characterized by deleterious germline mutations in breast cancer susceptibility gene 1 (BRCA1) or breast cancer susceptibility gene 2 (BRCA2) (i.e., is germline BRCA1- or BRCA2-mutated). In some such embodiments, the BRCA1- or BRCA2-mutated cancer is prostate cancer, breast cancer, ovarian cancer, peritoneal cancer, fallopian tube cancer, or pancreatic cancer.

In some embodiments of the methods, combinations and uses described herein, the cancer is characterized by amplification or overexpression of CDK4, CDK6 or cyclin D1 (CCND1). In some embodiments, the cancer is RB-positive or RB-proficient.

In some embodiments of each of the methods, combinations, uses described herein, the cancer is resistant to a therapeutic agent or class of agents, such as a standard of care agent or class for the particular cancer. In some embodiments of each of the methods, combinations, uses described herein, the cancer is characterized by innate or acquired resistance to a therapeutic agent or class of agents. In some such embodiments, the cancer is resistant to treatment with antiandrogens, taxanes, platinum agents, aromatase inhibitors, selective estrogen receptor degraders (SERDs), selective estrogen receptor modulators (SERMs), or CDK4/6 inhibitors.

In some embodiments of each of the methods, combinations and uses described herein, the cancer is resistant to treatment with an antiandrogen. In some embodiments wherein the cancer is resistant to treatment with an antiandrogen, the underlying resistance mechanism of the cancer is selected from the group consisting of AR activating mutations; splice variants not resistant to antiandrogen therapy; and other by pass mechanisms. In some such embodiments, the cancer is resistant to treatment with enzalutamide or abiraterone, or a pharmaceutically acceptable salt or solvate thereof. In other embodiments, the cancer is resistant to treatment with an androgen receptor inhibitor. In some such embodiments, the cancer is resistant to treatment with an androgen receptor inhibitor selected from the group consisting of enzalutamide, desmethyl enzalutamide, darolutamide, and apalutamide, or a pharmaceutically acceptable salt or solvate thereof. In some such embodiments the cancer is resistant to treatment with enzalutamide, or a pharmaceutically acceptable salt or solvate thereof.

In some embodiments of each of the methods, combinations and uses described herein, the cancer is resistant to treatment with a taxane (i.e., the cancer is a taxane resistant cancer).

In some embodiments of each of the methods, combinations and uses described herein, the cancer is resistant to treatment with a platinum agent (i.e., the cancer is a platinum resistant cancer).

In some embodiments of each of the methods, combinations and uses described herein, the cancer is resistant to treatment with an aromatase inhibitor, a SERD, or a SERM.

In some embodiments of each of the methods, combinations and uses described herein, the cancer is resistant to treatment with a CDK4 inhibitor or a CDK4/6 inhibitor. In some such embodiments, the cancer is resistant to treatment with a CDK4/6 inhibitor selected from the group consisting of palbociclib, ribociclib or abemaciclib, or a pharmaceutically acceptable salt thereof. In some such embodiments the cancer is resistant to treatment with palbociclib, or a pharmaceutically acceptable salt thereof.

In some embodiments of each of the methods, combinations, and uses described herein, the cancer is refractory, i.e., the cancer does not respond at all to treatment with a therapeutic agent or class (including a standard of care agent or class for the particular cancer) or initially responds but starts to grow again in a very short period of time.

In some embodiments of each of the methods, combinations and uses described herein, the cancer is prostate cancer. In some such embodiments, the prostate cancer is androgen-dependent. In some such embodiments, the prostate cancer is AR+ prostate cancer.

In some embodiments of the methods, combinations and uses described herein, the prostate cancer is advanced or metastatic prostate cancer. In some embodiments of the methods, combinations and uses described herein, the prostate cancer is early stage or non-metastatic prostate cancer.

In some embodiments of the methods, combinations and uses described herein, the prostate cancer is BRCA1- or BRCA2-mutated prostate cancer.

In some embodiments, the prostate cancer is castration resistant prostate cancer. In other embodiments, the prostate cancer is castration sensitive prostate cancer. In some embodiments of each of the methods, combinations and uses described herein, the prostate cancer is metastatic prostate cancer (mPC). In some such embodiments, the mPC is metastatic castration resistant prostate cancer (mCRPC). In other such embodiments, the mPC is metastatic castration-sensitive prostate cancer (mCSPC). In some embodiments of each of the methods, combinations and uses described herein, the prostate cancer is non-metastatic prostate cancer (nmPC). In some such embodiments, the nmPC is non-metastatic castration resistant prostate cancer (nmCRPC). In some such embodiments, the nmPC is non-metastatic castration sensitive prostate cancer (nmCSPC).

In some embodiments of each of the foregoing, the cancer is prostate cancer and the treatment achieved by a combination of the invention is measured by the time to PSA progression, the time to initiation of cytotoxic chemotherapy, or the proportion of patients with PSA response greater than or equal to 50%.

In some embodiments of the methods, combinations and uses described herein, the prostate cancer is refractory or resistant to treatment with, or has progressed on, one or more standard of care agents. In some such embodiments, the prostate cancer is refractory or resistant to treatment with, or has progressed on, antiandrogen therapy. In other embodiments, the prostate cancer is refractory or resistant to treatment with, or has progressed on, antineoplastic chemotherapeutic agents such as taxanes, platinum agents, anthracyclines or anti-metabolites.

In some such embodiments, the prostate cancer is refractory or resistant to treatment with an antiandrogen. In some such embodiments, the prostate cancer is refractory or resistant to treatment with enzalutamide or abiraterone, or a pharmaceutically acceptable salt or solvate thereof. In some embodiments of each of the foregoing, the prostate cancer is refractory or resistant to treatment with an androgen receptor inhibitor. In some such embodiments, the prostate cancer is refractory or resistant to treatment with enzalutamide, or a pharmaceutically acceptable salt or solvate thereof.

In some such embodiments, the prostate cancer is resistant to treatment with an antiandrogen. In some such embodiments, the prostate cancer is resistant to treatment with enzalutamide or abiraterone, or a pharmaceutically acceptable salt or solvate thereof. In some embodiments of each of the foregoing, the prostate cancer is resistant to treatment with an androgen receptor inhibitor. In some such embodiments, the prostate cancer is resistant to treatment with enzalutamide, or a pharmaceutically acceptable salt or solvate thereof.

In some embodiments wherein the cancer is prostate cancer, the methods, combinations and uses described herein further comprise an additional anti-cancer agent. In some such embodiments, the additional anti-cancer agent is androgen deprivation therapy (ADT). In some embodiments, the cancer is prostate cancer and the subject is further treated with androgen deprivation therapy (ADT) or a bilateral orchiectomy. In some such embodiments, the ADT is selected from the group consisting of a gonadotropin releasing hormone (GnRH) agonist and a gonadotropin releasing hormone (GnRH) antagonist. In some such embodiments, the ADT is selected from the group consisting of leuprolide, buserelin, gonadorelin, goserelin, histrelin, nafarelin, triptorelin, deslorelin, fertirelin, abarelix, cetrorelix, degarelix, ganirelix, ozarelix, elagolix, relugolix and linzagolix, or a pharmaceutically acceptable salt thereof.

In some embodiments of each of the methods, combinations and uses described herein, the cancer is breast cancer. In some such embodiments, the breast cancer is androgen-dependent breast cancer. In some embodiments, the breast cancer is AR+ breast cancer.

In some embodiments of the methods, combinations and uses described herein, the breast cancer is advanced or metastatic breast cancer. In some embodiments of the methods, combinations and uses described herein, the breast cancer is early stage or non-metastatic breast cancer.

In some embodiments of the methods, combinations and uses described herein, the breast cancer is characterized by amplification or overexpression of CDK4, CDK6 or cyclin D1 (CCND1). In some embodiments, the breast cancer is characterized as RB-positive, RB-proficient, or RB wild type.

In some embodiments of the methods, combinations and uses described herein, the breast cancer is BRCA1- or BRCA2-mutated breast cancer.

In some embodiments of the methods, combinations and uses described herein, the breast cancer is PIK3CA-mutated cancer breast cancer.

In some embodiments of the methods, combinations and uses described herein, the breast cancer is refractory or resistant to treatment with, or has progressed on, one or more standard of care agents. In some such embodiments, the breast cancer is refractory or resistant to treatment with, or has progressed on, an antiestrogen, such as an aromatase inhibitor, SERD, or a SERM. In some such embodiments, the breast cancer is refractory or resistant to treatment with, or has progressed on, a CDK4/6 inhibitor, such as palbociclib or a pharmaceutically acceptable salt thereof. In other embodiments, the breast cancer is refractory or resistant to treatment with, or has progressed on, treatment with antineoplastic chemotherapeutic agents such as taxanes, platinum agents, anthracyclines or anti-metabolites.

In some embodiments of each of the methods, combinations and uses described herein, the breast cancer is hormone receptor (HR)-positive (HR+) breast cancer, i.e., the breast cancer is estrogen receptor (ER)-positive (ER+) and/or progesterone receptor (PR)-positive (PR+).

In some embodiments, the breast cancer is hormone receptor (HR)-negative (HR−), i.e., the breast cancer is estrogen receptor (ER)-negative (ER−) and progesterone receptor (PR)-negative (PR−).

In some embodiments, the breast cancer is human epidermal growth factor receptor 2 (HER2)-positive (HER2+).

In some embodiments, the breast cancer is human epidermal growth factor receptor 2 (HER2)-negative (HER2−). In some such embodiments, the breast cancer is is estrogen receptor alpha (ERa)-negative.

In some embodiments, the breast cancer is triple negative breast cancer (TNBC), i.e., the breast cancer is ER−, PR− and HER2−.

In some embodiments, the breast cancer is selected from the group consisting of HR+/HER2− breast cancer, HR+/HER2+ breast cancer, HR−/HER2+ breast cancer, and triple negative breast cancer (TNBC). In some such embodiments, the breast cancer is androgen-dependent or AR+ breast cancer. In some such embodiments, the breast cancer is BRCA1- or BRCA2-mutated breast cancer.

In some embodiments, the breast cancer is HR+/HER2− breast cancer. In some such embodiments, the HR+/HER2− breast cancer is advanced or metastatic HR+/HER2− breast cancer. In some embodiments, the HR+/HER2− breast cancer is early or non-metastatic HR+/HER2− breast cancer.

In some embodiments, the HR+/HER2− breast cancer is characterized by amplification or overexpression of CDK4, CDK6 or cyclin D1 (CCND1). In some embodiments, the HR+/HER2− breast cancer is characterized as RB-positive, RB-proficient, or RB wild type.

In some embodiments, the HR+/HER2− breast cancer is BRCA1- or BRCA2-mutated breast cancer.

In some embodiments, the HR+/HER2− breast cancer is PIK3CA-mutated cancer breast cancer

In some such embodiments, the HR+/HER2− breast cancer is refractory or resistant to treatment with, or has progressed on, a standard of care agent, e.g., an antiestrogen such as an aromatase inhibitor, a SERD, or a SERM. In some such embodiments, the HR+/HER2− breast cancer is refractory or resistant to treatment with, or has progressed on, a CDK4/6 inhibitor, such as palbociclib or a pharmaceutically acceptable salt thereof.

In some such embodiments, the HR+/HER2− breast cancer is refractory or resistant to treatment an antiestrogen such as an aromatase inhibitor, a SERD, or a SERM. In some such embodiments, the HR+/HER2− breast cancer is refractory or resistant to treatment with a CDK4/6 inhibitor, such as palbociclib or a pharmaceutically acceptable salt thereof. In some such embodiments, the HR+/HER2− breast cancer is refractory or resistant to treatment with a CDK4/6 inhibitor, such as palbociclib or a pharmaceutically acceptable salt thereof, in further combination with an antiestrogen, e.g., letrozole or fulvestrant.

In some such embodiments, the HR+/HER2− breast cancer is resistant to treatment an antiestrogen such as an aromatase inhibitor, a SERD, or a SERM. In some such embodiments, the HR+/HER2− breast cancer is resistant to treatment with a CDK4/6 inhibitor, such as palbociclib or a pharmaceutically acceptable salt thereof. In some such embodiments, the HR+/HER2− breast cancer is resistant to treatment with a CDK4/6 inhibitor, such as palbociclib or a pharmaceutically acceptable salt thereof, in further combination with an antiestrogen, e.g., letrozole or fulvestrant.

In some embodiments, the breast cancer is HR+/HER2+ breast cancer. In some embodiments, the breast cancer is HR−/HER2+ breast cancer.

In some embodiments wherein the breast cancer is HR+, the methods, combinations and uses described herein further comprise an additional anti-cancer agent. In some such embodiments, the additional anti-cancer agent is an antiestrogen, such as an aromatase inhibitor, a SERD, or a SERM. In some such embodiments, the antiestrogen is letrozole or fulvestrant. In some such embodiments, the additional anti-cancer agent is a CDK4/6 inhibitor, such as palbociclib or a pharmaceutically acceptable salt thereof. In some such embodiments, the additional anti-cancer agent is a CDK4/6 inhibitor, such as palbociclib or a pharmaceutically acceptable salt thereof, in further combination with an antiestrogen, e.g., letrozole or fulvestrant. In some such embodiments, the additional anti-cancer agent is a PI3K inhibitor, e.g., alepelisib.

In some embodiments wherein the breast cancer is HER2+, the methods, combinations and uses described herein further comprise an additional anti-cancer agent. In some such embodiments, the additional anti-cancer agent is a HER2-targeted agent, e.g., trastuzumab emtansine, fam-trastuzumab deruxtecan, pertuzumab, lapatinib, neratinib or tucatinib, or an agent targeting the PI3K/AKT/mTOR molecular pathway, e.g., ipatasertib.

In some embodiments, the breast cancer is triple negative breast cancer (TNBC). In some embodiments, the TNBC is androgen-dependent or AR+ TNBC. In some such embodiments, the TNBC is RN+ or RB-proficient. In some such embodiments, the TNBC is AR+, RB+ or AR+, RB-proficient TNBC.

In some such embodiments, the TNBC is locally recurrent/advanced or metastatic TNBC. In some such embodiments, the TNBC is advanced or metastatic TNBC. In some such embodiments, the TNBC is early or non-metastatic TNBC.

In some embodiments, the TNBC is characterized by amplification or overexpression of CDK4, CDK6 or cyclin D1 (CCND1).

In some embodiments, the TNBC is BRCA1- or BRCA2-mutated TNBC.

In some embodiments, the TNBC is refractory or resistant to treatment with, or has progressed on, a standard of care agent, e.g., an antineoplastic chemotherapeutic agent such as a taxane, platinum agent, anthracycline or anti-metabolite.

In some embodiments of each of the methods, combinations and uses described herein, the cancer is lung cancer. In some embodiments, the lung cancer is non-small cell lung cancer (NSCLC). In some embodiments, the lung cancer is small cell lung cancer (SCLC). In some such embodiments, the lung cancer is advanced or metastatic lung cancer.

In some embodiments of each of the methods, combinations and uses described herein, the cancer is liver cancer. In some such embodiments the liver cancer is hepatocellular carcinoma (HCC). In some such embodiments, the liver cancer is advanced or metastatic liver cancer.

In some embodiments of each of the methods, combinations and uses described herein, the cancer is kidney cancer. In some such embodiments the kidney cancer is renal cell carcinoma (RCC). In some such embodiments, the kidney cancer is advanced or metastatic kidney cancer.

In some embodiments of each of the methods, combinations and uses described herein, the cancer is bladder cancer. In some such embodiments the bladder cancer is a urothelial carcinoma, including an upper urinary tract urothelial carcinoma (UUTUC). In some such embodiments, the bladder cancer is advanced or metastatic bladder cancer.

In some embodiments of each of the methods, combinations and uses described herein, the cancer is ovarian cancer, including epithelial ovarian cancer (EOC). In some such embodiments, the ovarian cancer is advanced or metastatic ovarian cancer.

In some embodiments of each of the methods, combinations and uses described herein, the cancer is peritoneal cancer, including primary peritoneal cancer (PPC). In some such embodiments, the peritoneal cancer is advanced or metastatic peritoneal cancer.

In some embodiments of each of the methods, combinations and uses described herein, the cancer is fallopian tube cancer. In some such embodiments, the fallopian tube cancer is advanced or metastatic fallopian tube cancer.

In some embodiments of each of the methods, combinations and uses described herein, the cancer is cervical cancer. In some such embodiments, the cervical cancer is advanced or metastatic cervical cancer.

In some embodiments of each of the methods, combinations and uses described herein, the cancer is uterine cancer, including endometrial cancer. In some such embodiments, the uterine cancer is advanced or metastatic uterine cancer.

In some embodiments of each of the methods, combinations and uses described herein, the cancer is pancreatic cancer. In some such embodiments, the pancreatic cancer is advanced or metastatic pancreatic cancer. In some such embodiments, the pancreatic cancer is resistant to antineoplastic chemotherapeutic agents such as taxanes, platinum agent, anthracyclines or anti-metabolites. In some such embodiments, the pancreatic cancer is resistant to gemcitabine or nab-paclitaxel.

In some embodiments of each of the methods, combinations and uses described herein, the cancer is stomach cancer. In some such embodiments, the stomach cancer is advanced or metastatic stomach cancer.

In some embodiments of each of the methods, combinations and uses described herein, the cancer is colorectal cancer. In some such embodiments, the colorectal cancer is advanced or metastatic colorectal cancer.

In some embodiments of each of the methods, combinations and uses described herein, the cancer is esophageal cancer. In some such embodiments, the esophageal cancer is advanced or metastatic esophageal cancer.

In some embodiments of each of the methods, combinations and uses described herein, the cancer is head and neck cancer. In some such embodiments, the head and neck cancer is advanced or metastatic head and neck cancer. In some such embodiments, the head and neck cancer is squamous cell carcinoma of the head and neck (SCCHN), thyroid cancer, or salivary gland cancer. In some such embodiments the head and neck cancer is salivary gland cancer.

In some embodiments of each of the methods, combinations and uses described herein, the cancer is testicular cancer. In some such embodiments, the testicular cancer is advanced or metastatic testicular cancer.

In some embodiments of each of the methods, combinations and uses described herein, the cancer is adrenal cancer. In some such embodiments, the adrenal cancer is advanced or metastatic adrenal cancer.

In some embodiments of each of the methods, combinations and uses described herein, the cancer is skin cancer. In some such embodiments, the skin cancer is basal cell carcinoma or melanoma. In some such embodiments, the skin cancer is advanced or metastatic skin cancer.

In some embodiments of each of the methods, combinations and uses described herein, the cancer is brain cancer. In some such embodiments, the brain cancer is astrocytoma, meningioma, or glioblastoma. In some such embodiments, the brain cancer is advanced or metastatic brain cancer.

In some embodiments of each of the methods, combinations and uses described herein, the cancer is sarcoma. In some such embodiments, the sarcoma is osteosarcoma or liposarcoma

In some embodiments of each of the methods, combinations and uses described herein, the cancer is lymphoma. In some such embodiments, the lymphoma is mantle cell lymphoma (MCL).

### Pharmaceutical Compositions, Medicaments and Kits

In one embodiment, this invention relates to a pharmaceutical composition comprising COMPOUND A, or a pharmaceutically acceptable salt thereof, and an antiandrogen, or a pharmaceutically acceptable salt or solvate thereof, and a pharmaceutically acceptable carrier.

In one embodiment, this invention relates to a pharmaceutical composition comprising COMPOUND A, or a pharmaceutically acceptable salt thereof, and an androgen receptor inhibitor, or a pharmaceutically acceptable salt or solvate thereof, and a pharmaceutically acceptable carrier.

The present invention further provides pharmaceutical compositions, medicaments and kits comprising a compound of Formula (I), having the structure:

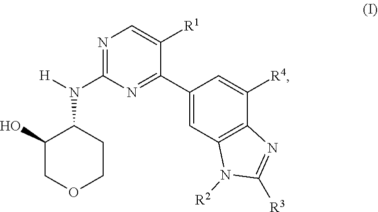

or a pharmaceutically acceptable salt thereof, wherein:
R¹ is H, F or Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy.

In another aspect, the invention provides a pharmaceutical composition comprising a compound of Formula (I) or a pharmaceutically acceptable salt thereof, an antiandrogen or a pharmaceutically acceptable salt thereof, and a pharmaceutically acceptable carrier or excipient. In some embodiments of this aspect, the pharmaceutical composition further comprises an additional anti-cancer agent (e.g., ADT).

In another aspect, the invention provides a first pharmaceutical composition comprising a compound of Formula (I) or a pharmaceutically acceptable salt thereof, and a pharmaceutically acceptable carrier or excipient, and a second pharmaceutical composition comprising an antiandrogen or a pharmaceutically acceptable salt thereof, and a pharmaceutically acceptable carrier or excipient, wherein the first and second pharmaceutical compositions are administered sequentially, simultaneously or concurrently. Some embodiments of this aspect further comprise a third pharmaceutical composition comprising an additional anti-cancer agent (e.g., ADT) and a pharmaceutically acceptable carrier or excipient, wherein the first, second and third pharmaceutical compositions are administered sequentially, simultaneously or concurrently.

In another aspect, the invention provides a combination comprising a compound of Formula (I) or a pharmaceutically acceptable salt thereof, and an antiandrogen or a pharmaceutically acceptable salt thereof, for use in the manufacture of a medicament for treating cancer in a subject. In another aspect, the invention provides use of a combination comprising a compound of Formula (I) or a pharmaceutically acceptable salt thereof, and an antiandrogen or a pharmaceutically acceptable salt thereof, in the manufacture of a medicament for treating cancer in a subject. In some embodiments of these aspects, the combination further comprises an additional anti-cancer agent (e.g., ADT) for use in the manufacture of a medicament.

In another aspect, the invention provides a compound of Formula (I) or a pharmaceutically acceptable salt thereof for use in the manufacture of a medicament for treating cancer, wherein the medicament is adapted for use in combination with an antiandrogen or a pharmaceutically acceptable salt thereof. In another aspect, the invention provides a compound of Formula (I) or a pharmaceutically acceptable salt thereof for use in the manufacture of a medicament for treating cancer, wherein the medicament is adapted for use in combination with an antiandrogen or a pharmaceutically acceptable salt thereof, and an additional anti-cancer agent (e.g., ADT). In another aspect, the invention provides use of a compound of Formula (I) or a pharmaceutically acceptable salt thereof, in the manufacture of a medicament for treating cancer, wherein the medicament is adapted for use in combination with an antiandrogen or a pharmaceutically acceptable salt thereof. In another aspect, the invention provides use of a compound of Formula (I) or a pharmaceutically acceptable salt thereof, in the manufacture of a medicament for treating cancer, wherein the medicament is adapted for use in combination with an antiandrogen or a pharmaceutically acceptable salt thereof, and an additional anti-cancer agent (e.g., ADT).

In some embodiments of the pharmaceutical compositions and medicaments described herein, the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof. In some such embodiments, the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A).

In some embodiments of the pharmaceutical compositions and medicaments described herein, the antiandrogen is enzalutamide or a pharmaceutically acceptable salt thereof.

In some embodiments of each of pharmaceutical compositions and medicaments described herein, the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof, and the antiandrogen is enzalutamide or a pharmaceutically acceptable salt thereof. In some such embodiments, the compound of Formula (I) is (1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A), and the antiandrogen is enzalutamide or a pharmaceutically acceptable salt thereof.

In another aspect, the invention provides a kit comprising a first container, a second container and a package insert, wherein the first container comprises at least one dose of a compound of Formula (I) or a pharmaceutically acceptable salt thereof, as further described herein; the second container comprises at least one dose of an antiandrogen or a pharmaceutically acceptable salt thereof; and the package insert comprises instructions for treating cancer in a subject using the medicaments. In another aspect, the invention provides a kit comprising a first container, a second container, a third container, and a package insert, wherein the first container comprises at least one dose of a compound of Formula (I) or a pharmaceutically acceptable salt thereof; the second container comprises at least one dose of an antiandrogen or a pharmaceutically acceptable salt thereof; the third container comprises at least one dose of an additional anti-cancer agent (e.g., ADT); and the package insert comprises instructions for treating cancer in a subject using the medicaments.

In some embodiments of the kits herein, the compound of Formula (I) is 1,5 anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof. In some such embodiments, the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2, 3-dideoxy-D-threo-pentitol (COMPOUND A). In some embodiments, the antiandrogen is enzalutamide or a pharmaceutically acceptable salt thereof.

In some embodiments of the kits herein, the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof, and the antiandrogen is enzalutamide or a pharmaceutically acceptable salt thereof. In some such embodiments of this aspect, the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A), and the antiandrogen is enzalutamide or a pharmaceutically acceptable salt thereof.

In some embodiments of the pharmaceutical compositions, medicaments, and kits comprising an additional anti-cancer agent, the additional anti-cancer agent is an androgen deprivation therapy (ADT) selected from the group consisting of a luteinizing hormone-releasing hormone (LHRH) agonist, a LHRH antagonist, a gonadotropin releasing hormone (GnRH) agonist and a GnRH antagonist. In some such embodiments, the androgen deprivation therapy is selected from the group consisting of leuprolide, buserelin, gonadorelin, goserelin, histrelin, nafarelin, triptorelin, deslorelin, fertirelin, abarelix, cetrorelix, degarelix, ganirelix, ozarelix, elagolix, relugolix and linzagolix, or a pharmaceutically acceptable salt thereof. In some such embodiments, the androgen deprivation therapy is selected from the group consisting of leuprolide, goserelin and degarelix.

In some embodiments of the pharmaceutical compositions, medicaments, and kits comprising an additional anti-cancer agent, the additional anti-cancer agent is an endocrine therapeutic agent, such as an aromatase inhibitor, a SERD, or a SERM. In some such embodiments, the antiestrogen is letrozole or fulvestrant.

The pharmaceutical compositions, medicaments and kits described herein may be useful for treating the cancers described above with respect to the methods, combinations and uses of the invention.

### Dosage Forms and Regimens

Each therapeutic agent of the methods and combination therapies of the present invention may be administered either alone, or in a medicament (also referred to herein as a pharmaceutical composition) which comprises the therapeutic agent and one or more pharmaceutically acceptable carriers, excipients, or diluents, according to pharmaceutical practice.

As used herein, the terms “combination” or “combination therapy” refer to the administration of two or more therapeutic agents of the combination therapy of the invention, either alone or in the form of a pharmaceutical composition or medicament, either sequentially, concurrently or simultaneously.

As used herein, the term “sequential” or “sequentially” refers to the administration of each therapeutic agent of the combination therapy of the invention, either alone or in a medicament, one after the other, wherein each therapeutic agent can be administered in any order. Sequential administration may be particularly useful when the therapeutic agents in the combination therapy are in different dosage forms, for example, one agent is a tablet and another agent is a sterile liquid, and/or the agents are administered according to different dosing schedules, for example, one agent is administered daily, and the second agent is administered less frequently such as weekly.

As used herein, the term “concurrently” refers to the administration of each therapeutic agent in the combination therapy of the invention, either alone or in separate medicaments, wherein the second therapeutic agent is administered immediately after the first therapeutic agent, but that the therapeutic agents can be administered in any order. In a preferred embodiment the therapeutic agents are administered concurrently.

As used herein, the term “simultaneous” refers to the administration of each therapeutic agent of the combination therapy of the invention in the same medicament, for example as a fixed dose combination comprising two or more drugs in a single dosage form.

In one embodiment of the present invention, COMPOUND A or a pharmaceutically acceptable salt thereof, is administered before administration of the antiandrogen or a pharmaceutically acceptable salt or solvate thereof.

In one embodiment of the present invention, COMPOUND A or a pharmaceutically acceptable salt thereof, is administered before administration of the androgen receptor inhibitor or a pharmaceutically acceptable salt or solvate thereof.

In one embodiment of the present invention, COMPOUND A or a pharmaceutically acceptable salt thereof, is administered before administration of enzalutamide or a pharmaceutically acceptable salt or solvate thereof.

In one embodiment of the present invention, the antiandrogen or a pharmaceutically acceptable salt or solvate thereof, is administered before administration of COMPOUND A or a pharmaceutically acceptable salt thereof.

In one embodiment of the present invention, the androgen receptor inhibitor or a pharmaceutically acceptable salt or solvate thereof, is administered before administration of COMPOUND A or a pharmaceutically acceptable salt thereof.

In one embodiment of the present invention, enzalutamide or a pharmaceutically acceptable salt or solvate thereof, is administered before administration of COMPOUND A or a pharmaceutically acceptable salt thereof.

In one embodiment of the present invention, COMPOUND A or a pharmaceutically acceptable salt thereof, is administered concurrently with the antiandrogen or a pharmaceutically acceptable salt or solvate thereof.

In one embodiment of the present invention, COMPOUND A or a pharmaceutically acceptable salt thereof, is administered concurrently with the androgen receptor inhibitor or a pharmaceutically acceptable salt or solvate thereof.

In one embodiment of the present invention, COMPOUND A or a pharmaceutically acceptable salt thereof, is administered concurrently with enzalutamide or a pharmaceutically acceptable salt or solvate thereof.

In one embodiment of the present invention, COMPOUND A or a pharmaceutically acceptable salt thereof, is administered simultaneously with the antiandrogen or a pharmaceutically acceptable salt or solvate thereof.

In one embodiment of the present invention, COMPOUND A or a pharmaceutically acceptable salt thereof, is administered simultaneously with the androgen receptor inhibitor or a pharmaceutically acceptable salt or solvate thereof.

In one embodiment of the present invention, COMPOUND A or a pharmaceutically acceptable salt thereof, is administered simultaneously with enzalutamide or a pharmaceutically acceptable salt or solvate thereof.

As will be understood by those skilled in the art, the combination therapy may be usefully administered to a subject during different stages of their treatment.

In some embodiments of each of the methods, combinations and uses described herein, the combination therapy is administered to a subject who is previously untreated, i.e. the subject is treatment naïve.

In some embodiments of each of the methods, combinations and uses described herein, the combination therapy is administered to a subject who has failed to achieve a sustained response after a prior therapy with a biotherapeutic or chemotherapeutic agent, i.e. the subject is treatment experienced.

In one embodiment of the present invention, the combination therapy is administered to a subject who has previously received androgen deprivation therapy, such as, but not limited to, LHRH agonist or LHRH antagonist.

In one embodiment of the present invention, the combination therapy is administered to a subject who has previously received androgen deprivation therapy, such as, but not limited to, luteinizing hormone-releasing hormone (LHRH) agonist or LHRH antagonist, or a gonadotropin-releasing hormone (GnRH) agonist or a GnRH antagonist. In some embodiments, the GnRH agonist is selected from the group consisting of leuprolide, buserelin, nafarelin, histrelin, goserelin, or deslorelin.

In some such embodiments, the combination therapy is administered to a subject who has previously received androgen deprivation therapy, but whose cancer has since progressed. In some such embodiments, the combination therapy is administered to a subject who has previously received a LHRH agonist or LHRH antagonist, but whose cancer has since progressed. In some such embodiments, the combination therapy is administered to a subject who has previously received a GnRH agonist or GnRH antagonist, but whose cancer has since progressed.

In one embodiment of the present invention, the combination therapy is administered to a subject who has previously undergone a bilateral orchiectomy. In some such embodiments, the combination therapy is administered to a subject who has previously undergone a bilateral orchiectomy, but whose cancer has since progressed.

In one embodiment of the present invention, the combination therapy is administered to a subject who has previously received an antiandrogen or taxane. In some such embodiments, the combination therapy is administered to a subject who has previously received an antiandrogen or a taxane, but whose cancer has since progressed.

In one embodiment of the present invention, the combination therapy is administered to a subject who has previously received an antiandrogen. In some such embodiments, the combination therapy is administered to a subject who has previously received an antiandrogen, but whose cancer has since progressed.

In one embodiment of the present invention, the combination therapy is administered to a subject who has previously received an androgen receptor inhibitor. In some such embodiments, the combination therapy is administered to a subject who has previously received an androgen receptor inhibitor, but whose cancer has since progressed.

In one embodiment of the present invention, the combination therapy is administered to a subject who has previously received enzalutamide or a pharmaceutically acceptable salt or solvate thereof. In some such embodiments, the combination therapy is administered to a subject who has previously received enzalutamide or a pharmaceutically acceptable salt or solvate thereof, but whose cancer has since progressed.

In one embodiment of the present invention, the combination therapy is administered to a subject who has previously received abiraterone acetate. In some such embodiments, the combination therapy is administered to a subject who has previously received abiraterone but whose cancer has since progressed.

In one embodiment of the present invention, the combination therapy is administered to a subject who has previously received a CDK4 or CDK4/6 inhibitor. In some such embodiments, the combination therapy is administered to a subject who has previously received a CDK4 or CDK4/6 inhibitor, but whose cancer has since progressed.

In one embodiment of the present invention, the combination therapy is administered to a subject who has previously received an antiestrogen. In some such embodiments, the combination therapy is administered to a subject who has previously received an antiestrogen, but whose cancer has since progressed.

In one embodiment of the present invention, the combination therapy is administered to a subject who has previously received a taxane. In some such embodiments, the combination therapy is administered to a subject who has previously received a taxane, but whose cancer has since progressed.

In one embodiment of the present invention, the combination therapy is administered to a subject diagnosed with prostate cancer, wherein the subject has a prostate specific antigen (PSA) level medically determined to be tumor-related.

In one embodiment of the present invention, the combination therapy is administered to a subject diagnosed with prostate cancer, wherein the subject has a prostate specific antigen (PSA) level of at least 2.0 ng/mL.

In one embodiment of the present invention, the combination therapy is administered to a subject diagnosed with prostate cancer, wherein the subject has a prostate specific antigen (PSA) level of at least 2.0 ng/mL, and wherein the prostate specific antigen (PSA) level has risen on at least two successive occasions at least 1 week apart.

In one embodiment of the present invention, the combination therapy is administered to a subject diagnosed with prostate cancer, wherein the subject has a prostate specific antigen(PSA) level which has doubled in ≤ 10 months.

In one embodiment of the present invention, the combination therapy is administered to a subject diagnosed with cancer, wherein the cancer has developed resistance to treatment with an antiandrogen.

In one embodiment of the present invention, the combination therapy is administered to a subject diagnosed with cancer, wherein the cancer has developed resistance to treatment with an antiandrogen or a taxane.

In one embodiment of the present invention, the combination therapy is administered to a subject diagnosed with cancer, wherein the cancer has developed resistance to treatment with an androgen receptor inhibitor.

In one embodiment of the present invention, the combination therapy is administered to a subject diagnosed with cancer, wherein the cancer has developed resistance to treatment with an antiandrogen and wherein the underlying resistance mechanism of the cancer is selected from the group consisting of AR activating mutations such as, but not limited to, AR F876 mutation; splice variants not resistant to antiandrogen therapy, such as, but not limited to, those associated with any neuroendocrine (NE) shift, such as, but not limited to, N-MYC upregulation, upregulation of AURKA, or loss of p53/RB; other by-pass mechanisms such as, but not limited to, glucocorticoid receptor (GR) upregulation.

In one embodiment of the present invention, the combination therapy is administered to a subject diagnosed with cancer, which cancer has developed resistance to treatment with an androgen receptor inhibitor and wherein the underlying resistance mechanism of the cancer is selected from the group consisting of AR activating mutations such as, but not limited to, AR F876 mutation; splice variants not resistant to antiandrogen therapy, such as, but not limited to, those associated with any neuroendocrine (NE) shift, such as, but not limited to, N-MYC upregulation, upregulation of AURKA, or loss of p53/RB; other by-pass mechanisms such as, but not limited to, glucocorticoid receptor (GR) upregulation.

In one embodiment of the present invention, the combination therapy is administered to a subject diagnosed with cancer, wherein the cancer has developed resistance to treatment with a CDK4 or CDK4/6 inhibitor.

In one embodiment of the present invention, the combination therapy is administered to a subject diagnosed with cancer, wherein the cancer has developed resistance to treatment with an antiestrogen. In one embodiment of the present invention, the combination therapy is administered to a subject diagnosed with cancer, wherein the cancer has developed resistance to treatment with an antiestrogen, wherein the antiestrogen is an aromatase inhibitor, a SERD or a SERM.

In some embodiments of each of the methods, combinations and uses described herein, the combination therapy may be administered prior to, or following surgery to remove a tumor, and/or may be used prior to, during or after radiation therapy, and/or may be used prior to, during or after chemotherapy.

In some embodiments of each of the methods, combinations and uses described herein, the invention relates to neoadjuvant therapy, adjuvant therapy, first-line therapy, second-line therapy, second-line or later therapy, or third-line or later therapy, in each case for treating cancer as further described herein. In each of the foregoing embodiments, the cancer may be localized, advanced or metastatic, and the intervention may occur at point along the disease continuum (i.e., at any stage of the cancer).

The efficacy of combinations described herein in certain tumors may be enhanced by combination with other approved or experimental cancer therapies, e.g., radiation, surgery, chemotherapeutic agents, targeted therapies, agents that inhibit other signaling pathways that are dysregulated in tumors, and other immune enhancing agents, such as PD-1 or PD-L1 antagonists and the like. The methods, combinations and uses of the current invention may further comprise one or more additional anti cancer agents.

Administration of combinations of the invention may be affected by any method that enables delivery of the compounds to the site of action. These methods include oral routes, intraduodenal routes, parenteral injection (including intravenous, subcutaneous, intramuscular, intravascular or infusion), topical, and rectal administration.

Dosage regimens may be adjusted to provide the optimum desired response. For example, a therapeutic agent of the combination therapy of the present invention may be administered as a single bolus, as several divided doses administered over time, or the dose may be proportionally reduced or increased as indicated by the exigencies of the therapeutic situation. It may be particularly advantageous to formulate a therapeutic agent in a dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention may be dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose may be readily established, and the effective amount providing a detectable therapeutic benefit to a subject may also be determined, as can the temporal requirements for administering each agent to provide a detectable therapeutic benefit to the subject. Accordingly, while certain dose and administration regimens are exemplified herein, these examples in no way limit the dose and administration regimen that may be provided to a subject in practicing the present invention.

It is to be noted that dosage values may vary with the type and severity of the condition to be alleviated and may include single or multiple doses. It is to be further understood that for any particular subject, specific dosage regimens should be adjusted over time according to the individual need and the professional judgment of the person administering or supervising the administration of the compounds or pharmaceutical compositions, taking into consideration factors such as the severity of the disorder or condition, the rate of administration, the disposition of the compound and the discretion of the prescribing physician. The dosage ranges set forth herein are exemplary only and are not intended to limit the scope or practice of the claimed compound or pharmaceutical composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens for administration of the chemotherapeutic agent are well-known in the relevant art and would be understood to be encompassed by the skilled artisan once provided the teachings disclosed herein

In some embodiments, at least one of the therapeutic agents in the combination therapy is administered using the same dosage regimen (dose, frequency and duration of treatment) that is typically employed when the agent is used as a monotherapy for treating the same cancer. In other embodiments, the subject received a lower total amount of at least one of the therapeutic agents in the combination therapy than when the same agent is used as a monotherapy, for example a lower dose of therapeutic agent, a reduced frequency of dosing and/or a shorter duration of dosing.

The dosage of a small molecule therapeutic agent, for example a compounds of Formula (I), an antiandrogen, or an androgen receptor inhibitor, is typically in the range of from about 0.001 to about 100 mg per kg body weight per day, preferably about 1 to about 35 mg/kg/day, in single or divided doses. For a 70 kg human, this would amount to about 0.01 to about 7 g/day, preferably about 0.02 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day. The dosage may be administered as a single dose (QD), or optionally may be subdivided into smaller doses, suitable for BID (twice daily), TID (three times daily) or QID (four times daily) administration. The dosage regimen may be adjusted to provide the optimal therapeutic response. For example, the dose may be proportionally reduced or increased as indicated by the exigencies of the therapeutic situation, including temporary or permanent dose reductions if required to ameliorate or prevent side effects.

In some embodiments herein, the androgen receptor inhibitor is enzalutamide, which is dosed in accordance with the approved label with a daily dose of 160 mg once daily. Dosage adjustments of enzalutamide, in accordance with full prescribing information may be readily determined by one of ordinary skill in the art, such as if the enzalutamide is to be dosed in concomitantly with a strong CYP2C8 inhibitor then the dose of enzalutamide should be reduced in accordance with the full prescribing information, such as to 80 mg once daily; or alternatively if the enzalutamide is to be dosed concomitantly with a CYP3A4 inducer then the dose of enzalutamide should be increased in accordance with the full prescribing information, such as to 240 mg daily, as can be determined by one of ordinary skill in the art.

In some embodiments herein, the antiandrogen is abiraterone acetate, which abiraterone acetate is dosed in accordance with the approved label with a daily dose of 1000 mg once daily in combination with prednisone 5 mg twice daily. Dosage adjustments of abiraterone acetate, in accordance with full prescribing information may be readily determined by one of ordinary skill in the art, such as if the abiraterone acetate is to be dosed concomitantly with a strong CYP3A4 inducer, then the dosage of abiraterone acetate may need to be increased for example to 1000 mg twice per day; if the abiraterone acetate is to be dosed concomitantly with a CYP2D6 substrate, then the dosage of abiraterone acetate may need to be reduced; if the abiraterone acetate is to be dosed to a subject or subject with baseline moderate hepatic impairment then the dose may need to be reduced, such as to 250 mg once daily; if the abiraterone acetate is to be dosed to a subject or subject who develops hepatotoxicity then the dose may need to be reduced, such as to 750 mg or 500 mg once daily.

Repetition of the administration or dosing regimens, or adjustment of the administration or dosing regimen may be conducted as necessary to achieve the desired treatment. A “continuous dosing schedule” as used herein is an administration or dosing regimen without dose interruptions, e.g. without days off treatment. Repetition of 21 or 28 day treatment cycles without dose interruptions between the treatment cycles is an example of a continuous dosing schedule. In an embodiment, the compounds of the combination of the present invention can be administered in a continuous dosing schedule.

In one embodiment of the present invention, COMPOUND A or a pharmaceutically acceptable salt thereof, and the antiandrogen, or a pharmaceutically acceptable salt of solvate thereof, are dosed in amounts which together are effective in treating the cancer.

In one embodiment of the present invention, COMPOUND A or a pharmaceutically acceptable salt thereof, and the androgen receptor inhibitor or a pharmaceutically acceptable salt of solvate thereof, are dosed in amounts which together are effective in treating the cancer.

In one embodiment of the present invention, COMPOUND A or a pharmaceutically acceptable salt thereof, and enzalutamide or a pharmaceutically acceptable salt of solvate thereof, are dosed in amounts which together are effective in treating the cancer.

In one embodiment of the present invention, COMPOUND A or a pharmaceutically acceptable salt thereof, and the antiandrogen or a pharmaceutically acceptable salt of solvate thereof, are dosed in amounts which together are synergistic.

In one embodiment of the present invention COMPOUND A or a pharmaceutically acceptable salt thereof, and the androgen receptor inhibitor or a pharmaceutically acceptable salt of solvate thereof, are dosed in amounts which together are synergistic.

In one embodiment of the present invention COMPOUND A or a pharmaceutically acceptable salt thereof, and enzalutamide or a pharmaceutically acceptable salt of solvate thereof, are dosed in amounts which together are synergistic.

In one embodiment of the present invention COMPOUND A, or a pharmaceutically acceptable salt thereof, and the antiandrogen or a pharmaceutically acceptable salt of solvate thereof, are dosed in a non-standard dosing regimen.

In one embodiment of the present invention, COMPOUND A or a pharmaceutically acceptable salt thereof, and the androgen receptor inhibitor or a pharmaceutically acceptable salt of solvate thereof, are dosed in a non-standard dosing regimen.

In one embodiment of the present invention, COMPOUND A or a pharmaceutically acceptable salt thereof, and enzalutamide or a pharmaceutically acceptable salt of solvate thereof, are dosed in a non-standard dosing regimen.

In one embodiment of the present invention COMPOUND A or a pharmaceutically acceptable salt thereof, and the antiandrogen or a pharmaceutically acceptable salt of solvate thereof, are dosed in a low dose regimen.

In one embodiment of the present invention COMPOUND A or a pharmaceutically acceptable salt thereof, and the androgen receptor inhibitor or a pharmaceutically acceptable salt of solvate thereof, are dosed in a low dose regimen.

In one embodiment of the present invention COMPOUND A or a pharmaceutically acceptable salt thereof, and enzalutamide or a pharmaceutically acceptable salt of solvate thereof, are dosed in a low dose regimen.

In some embodiments, the compound of Formula (I), or a corresponding amount of a pharmaceutically acceptable salt thereof, is administered at a daily dosage of from about 1 mg to about 1000 mg per day. In some embodiments, the compound of Formula (I), or a corresponding amount of a pharmaceutically acceptable salt thereof, is administered at a daily dosage from about 10 mg to about 500 mg per day, and in some embodiments, it is administered at a dosage of from about 25 mg to about 300 mg per day. In some embodiments it is administered at dosages of about 1, 2, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 260, 270, 275, 280, 290, 300, 325, 350, 375, 400, 425, 450, 475 or 500 mg on a QD, BID, TID or QID schedule.

In some embodiments, COMPOUND A, or a corresponding amount of a pharmaceutically acceptable salt thereof, is administered at a daily dosage of from about 1 mg to about 1000 mg per day. In some embodiments, COMPOUND A, or a corresponding amount of a pharmaceutically acceptable salt thereof, is administered at a daily dosage from about 10 mg to about 500 mg per day, and in some embodiments, it is administered at a dosage of from about 25 mg to about 300 mg per day. In some embodiments it is administered at dosages of about 1, 2, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 260, 270, 275, 280, 290, 300, 325, 350, 375, 400, 425, 450, 475 or 500 mg on a QD, BID, TID or QID schedule.

Repetition of the administration or dosing regimens, or adjustment of the administration or dosing regimen may be conducted as necessary to achieve the desired treatment. An “intermittent dosing schedule” as used herein refers to an administration or dosing regimen that includes a period of dose interruption, e.g. days off treatment. Repetition of 14 or 21 day treatment cycles with a 7 day treatment interruption between the treatment cycles is an example of an intermittent dosing schedule. Such schedules, with 2 or 3 weeks on treatment and 1 week off treatment, are sometimes referred to as a 2/1-week or 3/1-week treatment cycle, respectively. Alternatively, intermittent dosing may comprise a 7 day treatment cycle, with 5 days on treatment and 2 days off treatment.

A “continuous dosing schedule” as used herein is an administration or dosing regimen without dose interruptions, e.g. without days off treatment. Repetition of 21 or 28 day treatment cycles without dose interruptions between the treatment cycles is an example of a continuous dosing schedule.

In some embodiments, the compound of Formula (I) or a pharmaceutically acceptable salt thereof, and the antiandrogen are each administered in an intermittent dosing schedule. In other embodiments, the compound of Formula (I) or a pharmaceutically acceptable salt thereof, and the antiandrogen are each administered in a continuous dosing schedule.

In some such embodiments, COMPOUND A or a pharmaceutically acceptable salt thereof, and enzalutamide or a pharmaceutically acceptable salt thereof are each administered in an intermittent dosing schedule. In other embodiments, COMPOUND A or a pharmaceutically acceptable salt thereof, and enzalutamide or a pharmaceutically acceptable salt thereof are each administered in a continuous dosing schedule.

In still other embodiments, one of the compounds of Formula (I) or a pharmaceutically acceptable salt thereof, and the antiandrogen is administered in an intermittent dosing schedule (e.g., a 2/1-week or 3/1-week schedule) and the other is administered in a continuous dosing schedule. In some such embodiments, the compound of Formula (I) or a pharmaceutically acceptable salt thereof, is administered in an intermittent dosing schedule and the antiandrogen is administered in a continuous dosing schedule. In other such embodiments, the compound of Formula (I) or a pharmaceutically acceptable salt thereof, is administered in a continuous dosing schedule and the antiandrogen is administered in an intermittent dosing schedule.

In some such embodiments, one of COMPOUND A or a pharmaceutically acceptable salt thereof, and enzalutamide or a pharmaceutically acceptable salt thereof is administered in an intermittent dosing schedule (e.g., a 2/1-week or 3/1-week schedule) and the other is administered in a continuous dosing schedule. In some such embodiments, COMPOUND A or a pharmaceutically acceptable salt thereof is administered in an intermittent dosing schedule and enzalutamide or a pharmaceutically acceptable salt thereof is administered in a continuous dosing schedule. In other such embodiments, COMPOUND A or a pharmaceutically acceptable salt thereof is administered in a continuous dosing schedule and enzalutamide or a pharmaceutically acceptable salt thereof is administered in an intermittent dosing schedule.

In some embodiments of the present invention, the compound of Formula (I) or a pharmaceutically acceptable salt thereof, and the antiandrogen are dosed in amounts which together are effective in treating the cancer. In some such embodiments, COMPOUND A or a pharmaceutically acceptable salt thereof, and enzalutamide or a pharmaceutically acceptable salt thereof are dosed in amounts which together are effective in treating the cancer.

In some embodiments of the present invention, the compound of Formula (I) or a pharmaceutically acceptable salt thereof, and the antiandrogen are dosed in amounts which together are synergistic.

In some embodiments of the present invention, the compound of Formula (I) or a pharmaceutically acceptable salt thereof, and the antiandrogen are dosed in amounts which together are additive.

In some embodiments of the present invention, COMPOUND A or a pharmaceutically acceptable salt thereof, and the antiandrogen are dosed in amounts which together are synergistic. In some embodiments of the present invention, COMPOUND A or a pharmaceutically acceptable salt thereof and enzalutamide or a pharmaceutically acceptable salt thereof are dosed in amounts which together are additive.

In some embodiments of the present invention, COMPOUND A or a pharmaceutically acceptable salt thereof, and the antiandrogen are dosed in amounts which together are synergistic. In some embodiments of the present invention, COMPOUND A or a pharmaceutically acceptable salt thereof, and enzalutamide or a pharmaceutically acceptable salt thereof are dosed in amounts which together are additive.

### Pharmaceutical Compositions and Routes of Administration

A “pharmaceutical composition” refers to a mixture of one or more of the therapeutic agents described herein, or a pharmaceutically acceptable salt, solvate, hydrate or prodrug thereof as an active ingredient, and at least one pharmaceutically acceptable carrier or excipient. In some embodiments, the pharmaceutical composition comprises two or more pharmaceutically acceptable carriers and/or excipients.

As used herein, a “pharmaceutically acceptable carrier” refers to a carrier or diluent that does not cause significant irritation to an organism and does not abrogate the biological activity and properties of the active compound or therapeutic agent.

The pharmaceutical acceptable carrier may comprise any conventional pharmaceutical carrier or excipient. The choice of carrier and/or excipient will to a large extent depend on factors such as the particular mode of administration, the effect of the excipient on solubility and stability, and the nature of the dosage form.

In one embodiment, this invention relates to a pharmaceutical composition comprising COMPOUND A or a pharmaceutically acceptable salt thereof, and an antiandrogen or a pharmaceutically acceptable salt or solvate thereof, and a pharmaceutically acceptable carrier.

In one embodiment, this invention relates to a pharmaceutical composition comprising COMPOUND A or a pharmaceutically acceptable salt thereof, and an androgen receptor inhibitor or a pharmaceutically acceptable salt or solvate thereof, and a pharmaceutically acceptable carrier.

In one embodiment, this invention relates to a pharmaceutical composition comprising COMPOUND A or a pharmaceutically acceptable salt thereof, and enzalutamide or a pharmaceutically acceptable salt or solvate thereof, and a pharmaceutically acceptable carrier.

Suitable pharmaceutical carriers include inert diluents or fillers, water and various organic solvents (such as hydrates and solvates). The pharmaceutical compositions may, if desired, contain additional ingredients such as flavorings, binders, excipients and the like. Thus, for oral administration, tablets containing various excipients, such as citric acid may be employed together with various disintegrants such as starch, alginic acid and certain complex silicates and with binding agents such as sucrose, gelatin and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid pharmaceutical compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulation, solution or suspension, for parenteral injection as a sterile solution, suspension or emulsion, for topical administration as an ointment or cream, or for rectal administration as a suppository.

Exemplary parenteral administration forms include solutions or suspensions of an active compound in a sterile aqueous solution, for example, aqueous propylene glycol or dextrose solutions. Such dosage forms may be suitably buffered, if desired.

The pharmaceutical composition may be in unit dosage forms suitable for single administration of precise amounts.

Pharmaceutical compositions suitable for the delivery of the therapeutic agents of the combination therapies of the present invention, and methods for their preparation will be readily apparent to those skilled in the art. Such pharmaceutical compositions and methods for their preparation may be found, for example, in ‘Remington's Pharmaceutical Sciences’, 19th Edition (Mack Publishing Company, 1995), the disclosure of which is incorporated herein by reference in its entirety.

Therapeutic agents of the combination therapies of the invention may be administered orally. Oral administration may involve swallowing, so that the therapeutic agent enters the gastrointestinal tract, or buccal or sublingual administration may be employed by which the therapeutic agent enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films (including muco-adhesive), ovules, sprays and liquid formulations.

Liquid formulations include suspensions, solutions, syrups and elixirs. Such formulations may be used as fillers in soft or hard capsules and typically include a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Therapeutic agents of the combination therapies of the present invention may also be used in fast-dissolving, fast-disintegrating dosage forms such as those described in Expert Opinion in Therapeutic Patents, 11 (6), 981-986 by Liang and Chen (2001), the disclosure of which is incorporated herein by reference in its entirety.

For tablet dosage forms, the therapeutic agent may make up from 1 wt % to 80 wt % of the dosage form, more typically from 5 wt % to 60 wt % of the dosage form. In addition to the active agent, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinized starch and sodium alginate. Generally, the disintegrant may comprise from 1 wt % to 25 wt %, preferably from 5 wt % to 20 wt % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally include surface active agents, such as sodium lauryl sulfate and polysorbate 80, and glidants such as silicon dioxide and talc. When present, surface active agents are typically in amounts of from 0.2 wt % to 5 wt % of the tablet, and glidants typically from 0.2 wt % to 1 wt % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally are present in amounts from 0.25 wt % to 10 wt %, preferably from 0.5 wt % to 3 wt % of the tablet.

Other conventional ingredients include anti-oxidants, colorants, flavoring agents, preservatives and taste-masking agents.

Exemplary tablets may contain from about 1 wt % to about 80 wt % active agent, from about 10 wt % to about 90 wt % binder, from about 0 wt % to about 85 wt % diluent, from about 2 wt % to about 10 wt % disintegrant, and from about 0.25 wt % to about 10 wt % lubricant.

Tablet blends may be compressed directly or by roller to form tablets. Tablet blends or portions of blends may alternatively be wet-, dry-, or melt-granulated, melt congealed, or extruded before tableting. The final formulation may include one or more layers and may be coated or uncoated; or encapsulated.

The formulation of tablets is discussed in detail in “Pharmaceutical Dosage Forms: Tablets, Vol. 1”, by H. Lieberman and L. Lachman, Marcel Dekker, N.Y., N.Y., 1980 (ISBN 0-8247-6918-X), the disclosure of which is incorporated herein by reference in its entirety.

Capsules (made, for example, from gelatin or HPMC), blisters and cartridges for use in an inhaler or insufflator may be formulated to contain a powder mix of the therapeutic agent, a suitable powder base such as lactose or starch and a performance modifier such as 1-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

Solid formulations for oral administration may be formulated to be immediate and/or modified release. Modified release formulations include delayed-, sustained-, pulsed-, controlled-, targeted and programmed release.

Suitable modified release formulations are described in U.S. Pat. No. 6,106,864. Details of other suitable release technologies such as high energy dispersions and osmotic and coated particles may be found in Verma et al, Current Status of Drug Delivery Technologies and Future Directions, Pharmaceutical Technology On-line, (2001) 25:1-14. The use of chewing gum to achieve controlled release is described in WO 00/35298. The disclosures of these references are incorporated herein by reference in their entireties.

Therapeutic agents of the combination therapies of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including micro needle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilization, may readily be accomplished using standard pharmaceutical techniques well known to those skilled in the art.

The solubility of therapeutic agents used in the preparation of parenteral solutions may potentially be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus, therapeutic agents of the combination therapies of the invention may potentially be formulated as a solid, semi solid, or thixotropic liquid for administration as an implanted depot providing modified release of the active compound. Examples of such formulations include drug-coated stents and PGLA microspheres.

The therapeutic agents of the combination therapies of the present invention may conveniently be combined in the form of a kit suitable for coadministration of the pharmaceutical compositions. Such kits may comprise one or both of the active agents in the form of a pharmaceutical composition, which pharmaceutical composition comprises an active agent, or a pharmaceutically acceptable salt or solvate thereof, and a pharmaceutically acceptable carrier. The kit may contain means for separately retaining said pharmaceutical compositions, such as a container, divided bottle, or divided foil packet. An example of such a kit is the familiar blister pack used for the packaging of tablets, capsules and the like.

The kits described herein may be particularly suitable for administering different dosage forms, for example, oral and parenteral, for administering the separate pharmaceutical compositions at different dosage intervals, or for titrating the separate pharmaceutical compositions against one another. To assist compliance, the kit typically includes directions for administration and may be provided with a memory aid. The kit may further comprise other materials that may be useful in administering the medicaments, such as diluents, filters, IV bags and lines, needles and syringes, and the like.

### Additional Anti-Cancer Agents

The methods, combinations and uses of the present invention may further comprise one or more additional anti-cancer agents, such as the anti-angiogenesis agents, signal transduction inhibitors or antineoplastic agents described below, wherein the amounts together are effective in treating cancer. In some embodiments, the methods, combinations and uses of the present invention the additional anti-cancer agents may comprise a palliative care agent. Additional anti-cancer agents may include small molecules therapeutics and pharmaceutically acceptable salts or solvates thereof, therapeutic antibodies, antibody-drug conjugates (ADCs), proteolysis targeting chimeras (PROTACs), or antisense molecules.

In some such embodiments, the additional anti-cancer agent is selected from the group consisting of an anti-tumor agent, an anti-angiogenesis agent, a signal transduction inhibitor, and an antiproliferative agent. In some embodiments, the additional anti-cancer agent is selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, and endocrine therapeutic agents, such as antiandrogens, androgen deprivation therapy (ADT), and antiestrogens.

In some embodiments, the additional anti-cancer agent is an androgen deprivation therapy (ADT). In some such embodiment, the ADT is selected from the group consisting of a luteinizing hormone-releasing hormone (LHRH) agonist, a LHRH antagonist, a gonadotropin releasing hormone (GnRH) agonist and a GnRH antagonist. In one embodiment, the ADT is a LHRH agonist. In one embodiment the ADT is a LHRH antagonist. In one embodiment the ADT is a GnRH agonist. In one embodiment the ADT is a GnRH antagonist.

In some embodiments, the ADT is selected from the group consisting of leuprolide, buserelin, gonadorelin, goserelin, histrelin, nafarelin, triptorelin, deslorelin, fertirelin, abarelix, cetrorelix, degarelix, ganirelix, ozarelix, elagolix, relugolix, and linzagolix. In some such embodiments, the ADT is selected from the group consisting of leuprolide, goserelin and degaralix.

In one embodiment the ADT is leuprolide. In some embodiments the leuprolide is administered intramuscularly at a dose of about 7.5 mg every month, or about 22.5 mg every three months, or about 30 mg every four months. In some embodiments the leuprolide is administered subcutaneously at a dose of about 7.5 mg every month, or about 22.5 mg every three months, or about 30 mg every four months, or about 45 mg every six months, or about 65 mg every 12 months.

In one embodiment the ADT is goserelin. In some embodiments the goserelin is administered subcutaneously at a dose of about 3.6 mg every month, or about 10.8 mg every three months.

In one embodiment the ADT is degarelix. In some embodiments the degarelix is administered intramuscularly at an initial dose of about 240 mg, which initial dose may be optionally divided into several smaller doses, for example, two (2) doses of about 120 mg, followed by a maintenance dose of about 80 mg every month.

In some embodiments, the additional anti-cancer agent is an antiestrogen, wherein the antiestrogen is an aromatase inhibitor, a SERD, or a SERM. In some embodiments, the antiestrogen is an aromatase inhibitor. In some such embodiments, the aromatase inhibitor is selected from the group consisting of letrozole, anastrozole, and exemestane. In some such embodiments, the aromatase inhibitor is letrozole. In some embodiments, the antiestrogen is a SERD. In some such embodiments, the SERD is selected from the group consisting of fulvestrant, elacestrant (RAD-1901, Radius Health), SAR439859 (Sanofi), RG6171 (Roche), AZD9833 (AstraZeneca), AZD9496 (AstraZeneca), rintodestrant (G1 Therapeutics), ZN-c5 (Zentalis), LSZ102 (Novartis), D-0502 (Inventisbio), LY3484356 (Lilly), and SHR9549 (Jiansu Hengrui Medicine). In some such embodiments, the SERD is fulvestrant. In some embodiments, the antiestrogen is a SERM. In some such embodiments, the SERM is selected from the group consisting of tamoxifen, raloxifene, toremifene, lasofoxifene, bazedoxifene and afimoxifene. In some such embodiments, the SERM is tamoxifen or raloxifene.

In some embodiments, the methods, combinations and uses of the present invention further comprise one or more additional anti-cancer agents selected from the following:

Anti-angiogenesis agents include, for example, VEGF inhibitors, VEGFR inhibitors, TIE-2 inhibitors, PDGFR inhibitors, angiopoetin inhibitors, PKCp inhibitors, COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors.

Signal transduction inhibitors include, for example, kinase inhibitors (e.g., inhibitors of tyrosine kinases, serine/threonine kinases or cyclin dependent kinases), proteasome inhibitors, PI3K/AKT/mTOR pathway inhibitors, phosphoinositide 3-kinase (PI3K) inhibitors, isocitrate dehydrogenase 1 and 2 (IDH1 and IDH2) inhibitors, B-cell lymphoma 2 (BCL2) inhibitors, neurotrophin receptor kinase (NTRK) inhibitors, Rearranged during Transfection (RET) inhibitors, Notch inhibitors, PARP inhibitors, Hedgehog pathway inhibitors, and selective inhibitors of nuclear export (SINE).

Examples of signal transduction inhibitors inhibitors include, but are not limited to: acalabrutinib, afatinib, alectinib, alpelisib, axitinib, binimetinib, bortezomib, bosutinib, brigatinib, cabozantinib, carfilzomib, ceritinib, cobimetinib, copanlisib, crizotinib, dabrafenib, dacomitinib, dasatinib, duvelisib, enasidenib, encorafenib, entrectinib, erlotinib, gefitinib, gilteritinib, glasdegib, ibrutinib, idelalisib, imatinib, ipatasertib, ivosidenib, ixazomib, lapatinib, larotrectinib, lenvatinib, lorlatinib, midostaurin, neratinib, nilotinib, niraparib, olaparib, osimertinib, pazopanib, ponatinib, regorafenib, rucaparib, ruxolitinib, sonidegib, sorafenib, sunitinib, talazoparib, trametinib, vandetanib, vemurafenib, venetoclax, and vismodegib, or pharmaceutically acceptable salts and solvates thereof.

Antineoplastic agents include, for example, alkylating agents, platinum coordination complexes, cytotoxic antibiotics, antimetabolies, biologic response modifiers, histone deacetylate (HDAC) inhibitors, hormonal agents, monoclonal antibodies, growth factor inhibitors, taxanes, topoisomerase inhibitors, Vinca alkaloids and miscellaneous agents.

Alkylating agents include: altretamine, bendamustine, busulfan, carmustine, chlorambucil, cyclophosphamide, dacarbazine, ifosfamide, lomustine, mechlorethamine, melphalan, procarbazine, streptozocin, temozolomide, thiotepa, and trabectedin.

Platinum coordination complexes (also referred to herein as “platinum agents”) include: carboplatin, cisplatin, and oxaliplatin.

Cytotoxic antibiotics include: bleomycin, dactinomycin, daunorubicin, doxorubicin, epirubicin, idarubicin, mitomycin, mitoxantrone, plicamycin, and valrubicin.

Antimetabolites include: antifolates, such as methotrexate, pemetrexed, pralatrexate, and trimetrexate; purine analogues, such as azathioprine, cladribine, fludarabine, mercaptopurine, and thioguanine; and pyrimidine analogues such as azacitidine, capecitabine, cytarabine, decitabine, floxuridine, fluorouracil, gemcitabine, and trifluridine/tipracil.

Biologic response modifiers include: aldesleukin (IL-2), denileukin diftitox, and interferon gamma.

Histone deacetylase inhibitors include belinostat, panobinostat, romidepsin, and vorinostat.

Hormonal agents include antiandrogens, antiestrogens, gonadotropin releasing hormone (GnRH) analogues and peptide hormones. Examples of antiestrogens include: aromatase inhibitors, such as letrozole, anastrozole, and exemestane; SERDs, such as fulvestrant, elacestrant (RAD-1901, Radius Health), SAR439859 (Sanofi), RG6171 (Roche), AZD9833 (AstraZeneca), AZD9496 (AstraZeneca), rintodestrant (G1 Therapeutics), ZN-c5 (Zentalis), LSZ102 (Novartis), D-0502 (Inventisbio), LY3484356 (Lilly), SHR9549 (Jiansu Hengrui Medicine); and SERMs, such as tamoxifen, raloxifene, toremifene, lasofoxifene, bazedoxifene, afimoxifene. Examples of GnRH analogues include: degarelix, goserelin, histrelin, leuprolide, and triptorelin. Examples of peptide hormones include: lanreotide, octreotide, and pasireotide. Examples of antiandrogens include: abiraterone, apalutamide, bicalutamide, cyproterone, enzalutamide, flutamide, and nilutamide, and pharmaceutically acceptable salts and solvates thereof.

Monoclonal antibodies include: alemtuzumab, atezolizumab, avelumab, bevacizumab, blinatumomab, brentuximab, cemiplimab, cetuximab, daratumumab, dinutuximab, durvalumab, elotuzumab, gemtuzumab, inotuzumab ozogamicin, ipilimumab, mogamulizumab, moxetumomab pasudotox, necitumumab, nivolumab, ofatumumab, olaratumab, panitumumab, pembrolizumab, pertuzumab, ramucirumab, rituximab, tositumomab, and trastuzumab.

Taxanes include: cabazitaxel, docetaxel, paclitaxel and paclitaxel albumin-stabilized nanoparticle formulation (Nab-paclitaxel).

Topoisomerase inhibitors include: etoposide, irinotecan, teniposide, and topotecan.

Vinca alkaloids include: vinblastine, vincristine, and vinorelbine, and pharmaceutically acceptable salts thereof.

Miscellaneous antineoplastic agents include: asparaginase (pegaspargase), bexarotene, eribulin, everolimus, hydroxyurea, ixabepilone, lenalidomide, mitotane, omacetaxine, pomalidomide, tagraxofusp, telotristat, temsirolimus, thalidomide, and venetoclax.

In some embodiments, the additional anti-cancer agent is selected from the group consisting of: abiraterone acetate; acalabrutinib; ado-trastuzumab emtansine; afatinib dimaleate; afimoxifene; aldesleukin; alectinib; alemtuzumab; alpelisib; amifostine; anastrozole; apalutamide; aprepitant; arsenic trioxide; asparaginase erwinia chrysanthemi; atezolizumab; avapritinib; avelumab; axicabtagene ciloleucel; axitinib; azacitidine; AZD9833 (AstraZeneca); AZD9496 (AstraZeneca); bazedoxifene; belinostat; bendamustine hydrochloride; bevacizumab; bexarotene; bicalutamide; binimetinib; bleomycin sulfate; blinatumomab; bortezomib; bosutinib; brentuximab vedotin; brigatinib; cabazitaxel; cabozantinib-s-malate; calaspargase pegol-mknl; capecitabine; caplacizumab-yhdp; capmatinib hydrochloride; carboplatin; carfilzomib; carmustine; cemiplimab-rwlc; ceritinib; cetuximab; chlorambucil; cisplatin; cladribine; clofarabine; cobimetinib; copanlisib hydrochloride; crizotinib; cyclophosphamide; cytarabine; D-0502 (Inventisbio); dabrafenib mesylate; dacarbazine; dacomitinib; dactinomycin; daratumumab; daratumumab and hyaluronidase-fihj; darbepoetin alfa; darolutamide; dasatinib; daunorubicin hydrochloride; decitabine; defibrotide sodium; degarelix; denileukin diftitox; denosumab; dexamethasone; dexrazoxane hydrochloride; dinutuximab; docetaxel; doxorubicin hydrochloride; durvalumab; duvelisib; elacestrant; elotuzumab; eltrombopag olamine; emapalumab-lzsg; enasidenib mesylate; encorafenib; enfortumab vedotin-ejfv; entrectinib; enzalutamide; epirubicin hydrochloride; epoetin alfa; erdafitinib; eribulin mesylate; erlotinib hydrochloride; etoposide; etoposide phosphate; everolimus; exemestane; fam-trastuzumab deruxtecan-nxki; fedratinib hydrochloride; filgrastim; fludarabine phosphate; fluorouracil; flutamide; fostamatinib disodium; fulvestrant; gefitinib; gemcitabine hydrochloride; gemtuzumab ozogamicin; gilteritinib fumarate; glasdegib maleate; glucarpidase; goserelin acetate; granisetron; granisetron hydrochloride; hydroxyurea; ibritumomab tiuxetan; ibrutinib; idarubicin hydrochloride; idelalisib; ifosfamide; imatinib mesylate; imiquimod; inotuzumab ozogamicin; interferon alfa-2b recombinant; iobenguane 1-131; ipatasertib; ipilimumab; irinotecan hydrochloride; isatuximab-irfc; ivosidenib; ixabepilone; ixazomib citrate; lanreotide acetate; lapatinib ditosylate; larotrectinib sulfate; lasofoxifene; lenalidomide; lenvatinib mesylate; letrozole; leucovorin calcium; leuprolide acetate; lomustine; lorlatinib; LSZ102 (Novartis); lurbinectedin; LY3484356 (Lilly); megestrol acetate; melphalan; melphalan hydrochloride; mercaptopurine; methotrexate; midostaurin; mitomycin; mitoxantrone hydrochloride; mogamulizumab-kpkc; moxetumomab pasudotox-tdfk; necitumumab; nelarabine; neratinib maleate; nilotinib; nilutamide; niraparib tosylate monohydrate; nivolumab; obinutuzumab; ofatumumab; olaparib; omacetaxine mepesuccinate; ondansetron hydrochloride; osimertinib mesylate; oxaliplatin; paclitaxel; paclitaxel albumin-stabilized nanoparticle formulation; palifermin; palonosetron hydrochloride; pamidronate disodium; panitumumab; panobinostat; pazopanib hydrochloride; pegaspargase; pegfilgrastim; peginterferon alfa-2b; pembrolizumab; pemetrexed disodium; pemigatinib; pertuzumab; pexidartinib hydrochloride; plerixafor; polatuzumab vedotin-piiq; pomalidomide; ponatinib hydrochloride; pralatrexate; prednisone; procarbazine hydrochloride; propranolol hydrochloride; radium 223 dichloride; raloxifene hydrochloride; ramucirumab; rasburicase; ravulizumab-cwvz; recombinant interferon alfa-2b; regorafenib; RG6171 (Roche); rintodestrant; ripretinib; rituximab; rolapitant hydrochloride; romidepsin; romiplostim; rucaparib camsylate; ruxolitinib phosphate; sacituzumab govitecan-hziy; SAR439859 (Sanofi); selinexor; selpercatinib; selumetinib sulfate; SHR9549 (Jiansu Hengrui Medicine); siltuximab; sipuleucel-t; sonidegib; sorafenib tosylate; tagraxofusp-erzs; talazoparib tosylate; talimogene laherparepvec; tamoxifen citrate; tazemetostat hydrobromide; temozolomide; temsirolimus; thalidomide; thioguanine; thiotepa; tisagenlecleucel; tocilizumab; topotecan hydrochloride; toremifene; trabectedin; trametinib; trastuzumab; trastuzumab and hyaluronidase-oysk; trifluridine and tipiracil hydrochloride; tucatinib; uridine triacetate; valrubicin; vandetanib; vemurafenib; venetoclax; vinblastine sulfate; vincristine sulfate; vinorelbine tartrate; vismodegib; vorinostat; zanubrutinib; ziv-aflibercept; ZN-c5 (Zentalis); and zoledronic acid; or free base, pharmaceutically acceptable salt (including an alternative salt forms to the salts named above), or solvate forms of the foregoing; or combinations thereof.

Cancer cell spheroids have been reported to better recapitulate characteristics and cellular behavior of human in vivo tumors over conventional in vitro 2D monolayer cell cultures. Increases in cell-cell and cell-ECM interactions, local hypoxic areas, gradients of nutrients and pH, co-existence of proliferating and quiescent cells, altered cell morphology and altered drug penetrance due to cell compaction, as well as changes in the cellular metabolic profile, have been documented in multicellular tumor spheroid (MCTS) as compared to 2D monolayer cell culture (Zanoni et al., Anticancer drug discovery using multicellular tumor spheroid models, Expert Opin. Drug Discov. (2019) 14:289-301; Hamilton & Rath, Applicability of tumor spheroids for in vitro chemosensitivity assays, Expert Opin. Drug Metab. Toxicol. (2019) 15:15-23; Sant and Johnston, The production of 3D tumor spheroids for cancer drug discovery, Drug Discov. Today Technol. (2017) 23:27-36).

There is no change in cell density over the duration of an MCTS assay; this allows long-term treatment studies (1 month and beyond) without the need for cell detachment and cell reseeding procedures that can compromise assay performance and impact cellular physiology over time. Numerous examples of cancer drugs eliciting different activity in 3D versus 2D cell culture environments have been described (Karlsson et al., Loss of cancer drug activity in colon cancer HCT-116 cells during spheroid formation in a new 3-D spheroid cell culture system, Exp. Cell Res. (2012), 318:1577-85; Ekert et al., Three-dimensional lung tumor microenvironment modulates therapeutic compound responsiveness in vitro-implication for drug development, PLoS One (2014), 9: e92248; Wenzel et al., 3D high-content screening for the identification of compounds that target cells in dormant tumor spheroid regions, Exp. Cell Res. (2014), 323:131-43. Taken together, the observation of additive or synergistic anti-tumor cell growth inhibition in 3D/tumor cell spheroids in vitro provides increased confidence that the observed combinatorial benefit will translate to the clinical setting.

In some preferred embodiments, the invention provides:

E1. A method of treating cancer in a subject in need thereof comprising administering to the subject:
(a) an amount of a compound of Formula (I):

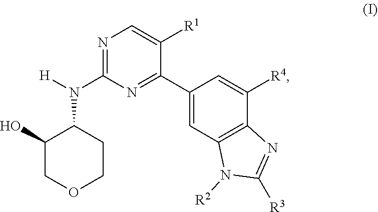

or a pharmaceutically acceptable salt thereof, wherein:
R¹ is H, F or Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy; and
(b) an amount of an antiandrogen;
wherein the amounts in (a) and (b) together are effective in treating cancer.

E2. The method of embodiment E1, wherein the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2, 3-dideoxy-D-threo-pentitol, or a pharmaceutically acceptable salt thereof.

E3. The method of embodiment E1 or E2, wherein the antiandrogen is selected from the group consisting of enzalutamide, N-desmethyl enzalutamide, darolutamide, apalutamide, and abiraterone, or a pharmaceutically acceptable salt or solvate thereof.

E4. The method of embodiment E3, wherein the antiandrogen is enzalutamide or a pharmaceutically acceptable salt or solvate thereof.

E5. The method of any one of embodiments E1 to E4, wherein the cancer is selected from the group consisting of prostate cancer, breast cancer, lung cancer, liver cancer, kidney cancer, bladder cancer, ovarian cancer, peritoneal cancer, fallopian tube cancer, cervical cancer, uterine cancer, pancreatic cancer, stomach cancer, colorectal cancer, esophageal cancer, head and neck cancer, testicular cancer, adrenal cancer, skin cancer, brain cancer, sarcoma, and lymphoma.

E6. The method of embodiment E5, wherein the cancer is prostate cancer.

E7. The method of embodiment E5 or E6, wherein the prostate cancer is metastatic prostate cancer (mPC).

E8. The method of embodiment E7, wherein the mPC is metastatic castration resistant prostate cancer (mCRPC).

E9. The method of embodiment E7, wherein the mPC is metastatic castration-sensitive prostate cancer (mCSPC).

E10. The method of embodiment E5 or E6, wherein the prostate cancer is non-metastatic prostate cancer (nmPC).

E11. The method of embodiment E10, wherein the nmPC is non-metastatic castration resistant prostate cancer (nmCRPC).

E12. The method of embodiment E10, wherein the nmPC is non-metastatic castration sensitive prostate cancer (nmCSPC).

E13. The method of any one of embodiments E5 to E12, wherein the prostate cancer is resistant to enzalutamide or abiraterone.

E14. The method of embodiment E5, wherein the cancer is breast cancer.

E15. The method of embodiment E14, wherein the breast cancer is hormone receptor (HR)-positive, human epidermal growth factor receptor 2 (HER2)-negative breast cancer.

E16. The method of embodiment E14, wherein the breast cancer is human epidermal growth factor receptor 2 (HER2)-positive breast cancer.

E17. The method of embodiment E14, wherein the breast cancer is triple negative breast cancer (TNBC).

E18. The method of any of embodiments E14 to E17, wherein the breast cancer is BRCA1- or BRCA2-mutated breast cancer.

E19. The method of embodiment E5, wherein the cancer is liver cancer.

E20. The method of embodiment E19, wherein the liver cancer is hepatocellular carcinoma (HCC).

E21. The method of any one of embodiments E1 to E20, wherein the compound of Formula (I) or a pharmaceutically acceptable salt thereof, and the antiandrogen or a pharmaceutically acceptable salt or solvate thereof are administered sequentially, simultaneously or concurrently.

E22. The method of any one of embodiment E1 to E21, further comprising administering to the subject: (c) an amount of an additional anti-cancer agent; wherein the amounts in (a), (b) and (c) together are effective in treating cancer.

E23. The method of embodiment E22, wherein the additional anti-cancer agent is selected from the group consisting of an anti-tumor agent, an anti-angiogenesis agent, a signal transduction inhibitor, an antiproliferative agent, and an androgen deprivation therapy (ADT).

E24. The method of embodiment E23, wherein the additional anti-cancer agent is an ADT.

E25. The method of embodiment E24, wherein the ADT is selected from the group consisting of a gonadotropin releasing hormone (GnRH) agonist and a gonadotropin releasing hormone (GnRH) antagonist.

E26. The method of embodiment E24, wherein the ADT is selected from the group consisting of leuprolide, buserelin, gonadorelin, goserelin, histrelin, nafarelin, triptorelin, deslorelin, fertirelin, abarelix, cetrorelix, degarelix, ganirelix, ozarelix, elagolix, relugolix and linzagolix, or a pharmaceutically acceptable salt thereof.

E27. The method of any one of embodiments E1 to E26, wherein the cancer is androgen dependent or androgen receptor (AR)-positive.

E28. The method of any one of embodiments E1 to E27, wherein the cancer is characterized by amplification or overexpression of CDK4, CDK6 or cyclin D1 (CCND1).

E29. The method of any one of embodiments E1 to E28, wherein the cancer is advanced or metastatic cancer.

E30. The method of any one of embodiments E1 to E29, wherein the subject is human.

E31. A combination comprising:
(a) a compound of Formula (I):

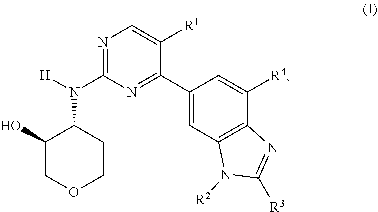

or a pharmaceutically acceptable salt thereof, wherein:
R¹ is H, F or Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy; and
(b) an antiandrogen;
wherein the combination of (a) and (b) is effective in treating cancer.

E32. The combination of embodiment E31, wherein the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol (COMPOUND A) or a pharmaceutically acceptable salt thereof, and the antiandrogen is enzalutamide or a pharmaceutically acceptable salt or solvate thereof.

E33. A combination for use in treating cancer comprising:
(a) a compound of Formula (I):

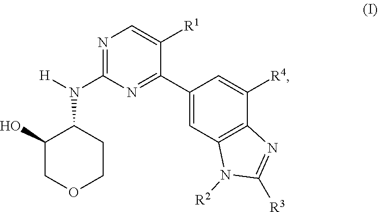

or a pharmaceutically acceptable salt thereof, wherein:
R¹ is H, F or Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy; and
(b) an antiandrogen.

E34. The combination of embodiment E33, wherein the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol or a pharmaceutically acceptable salt thereof, and the antiandrogen is enzalutamide or a pharmaceutically acceptable salt or solvate thereof.

E35. Use of a combination comprising:
(a) a compound of Formula (I):

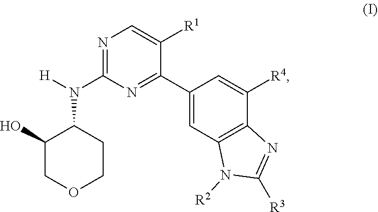

or a pharmaceutically acceptable salt thereof, wherein:
R¹ is H, F or Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy; and
(b) an antiandrogen;
wherein use of the combination is effective in treating cancer.

E36. The use of embodiment E35, wherein the compound of Formula (I) is 1,5-anhydro-3-({5-chloro-4-[4-fluoro-2-(2-hydroxypropan-2-yl)-1-(propan-2-yl)-1H-benzimidazol-6-yl]pyrimidin-2-yl}amino)-2,3-dideoxy-D-threo-pentitol or a pharmaceutically acceptable salt thereof, and the antiandrogen is enzalutamide or a pharmaceutically acceptable salt or solvate thereof.

These and other aspects of the invention, including the exemplary specific embodiments listed below, will be apparent from the teachings contained herein.

### EXAMPLES

Example 1—Multicellular Tumor Spheroid Growth Assay in Human AR+ Prostate Cancer Cells (LNCaP)

LNCaP prostate cancer cells were obtained from the ATCC and maintained in RPMI 1640 medium supplemented with 10% fetal bovine serum and penicillin-streptomycin as per ATCC guidelines. Cells were maintained in humidified incubator at 37° C. with 5% CO₂.

Spheroid assays were performed in 96 well ultralow attachment plates (ULA-96U) from Nexcelom & Thermo Fisher Scientific. One hundred twenty (120) LNCaP cells were dispensed in 200 μL of complete growth medium per well (n=10 to 12 wells per treatment group) of each ultralow attachment plate to allow formation of one spheroid per well with a diameter between 200 and 250 μm before the start of treatment (cell seeding numbers were previously optimized so that formed spheroids possessed this desired dimension). To aid spheroid formation, dispensed cells were centrifuged at 220×g for 6 minutes in the ultralow attachment plates and allowed to form compact spheroids for 4 days prior to the initiation of treatment. After spheroids were formed, 150 μL of medium was aspirated from each well without disturbing the spheroid, and fresh RPMI medium of the same volume was added containing single agent compounds (palbociclib, COMPOUND A, or enzalutamide), or selected combinations thereof. Final concentrations of each compound in the wells were: 30 or 100 nM for palbociclib; 100, 300 or 1000 nM for COMPOUND A; and 1000 nM for enzalutamide. DMSO (0.01%) was used as the vehicle control. DMSO and all compounds were diluted in cell medium. Medium and compounds were replenished twice per week, with 3 and 4-day intervals. Replenishment was executed by aspirating 150 μL of medium per well without disturbing the spheroid and then adding the same volume of premixed medium/compound solution to spheroids. In some cases, an extended phase of treatment was followed by a ‘recovery’ phase where medium was replenished without addition of compounds. Spheroid diameter was quantified immediately following each medium change twice a week (on every 3^{rd} or 4^{th} day) throughout the duration of the assay.

Growth of these MCTS was monitored over time to assess: (i) amplitude of response (SGI) and (ii) duration of response to single agents and combination treatments while on treatment.

### Data Analysis:

Average diameters of tumor spheroids were plotted in GraphPad Prism 8 and the area-under-curve (AUC) was calculated. AUC baseline was determined by the average tumor spheroid diameter at Day 0 in the vehicle (DMSO) controls. Spheroid growth inhibition, SGI, for all treatment arms was derived at the timepoint when the vehicle (DMSO) treated spheroids reach their maximal diameter (usually close to 1 mm but this can differ among cell lines); this corresponds to the last time point taken for the vehicle (DMSO) treated spheroids. SEM calculated based on n=10 to 12 wells per test group. Percent spheroid growth inhibition, or SGI %, was calculated as follows: SGI %=(1-AUC treatment/AUC DMSO)×100%.

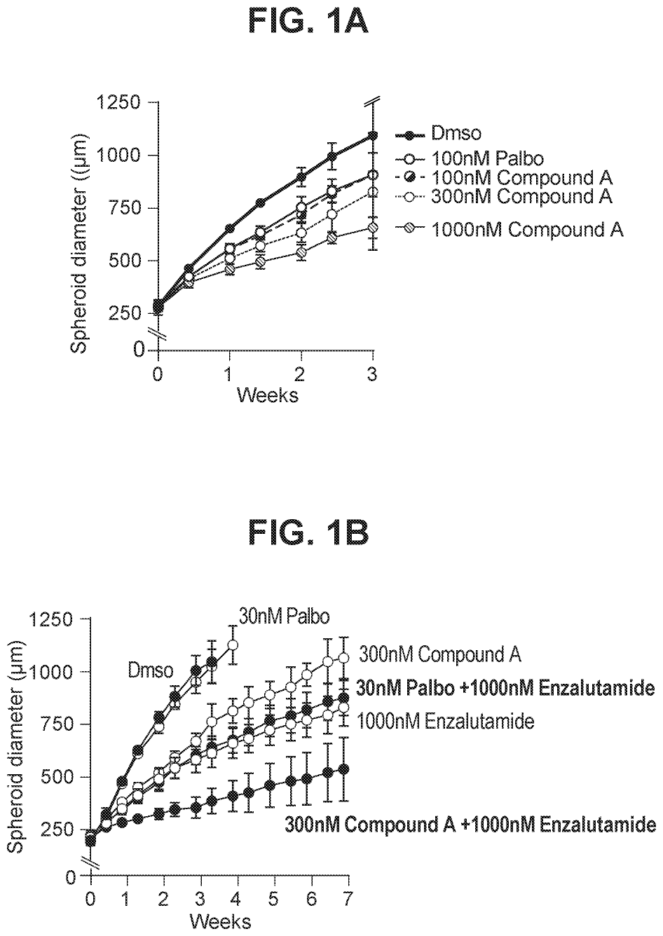

Spheroids were treated with increasing concentrations of 100, 300, or 1000 nM COMPOUND A and compared to palbociclib at the concentration of 100 nM (FIG. 1A). COMPOUND A showed dose-dependent inhibition of AR+ LNCaP prostate cancer spheroid growth (FIG. 1A). Addition of COMPOUND A to the AR inhibitor enzalutamide led to further inhibition of LNCaP spheroid growth (46% for 1000 nM enzalutamide alone versus 70% when 1000 nM enzalutamide was combined with 300 nM COMPOUND A) (FIG. 1B). By contrast, when 1000 nM enzalutamide was combined with 30 nM palbociclib there was no additive benefit (46% vs 47% spheroid growth inhibition) (FIG. 1B). Error bars represent standard error of measurement, SEM (n=10 to 12 wells per test group).

The percent of spheroid growth inhibition (SGI %) is indicated in Table 1 below.

TABLE 1
SGI % for single agent and combination treatments

| Treatment | SGI % |
|---|---|
| 30 nM palbociclib | 5 |
| 100 nM palbociclib | 24 |
| 100 nM COMPOUND A | 27 |
| 300 nM COMPOUND A | 38 |
| 1000 nM COMPOUND A | 53 |
| 1000 nM enzalutamide | 46 |
| 30 nM palbociclib/1000 nM enzalutamide | 47 |
| 300 nM COMPOUND A/1000 nM enzalutamide | 70 |

### Example 2—In Vitro Screen in C4-2 Human Prostate Cancer Cells

C4-2 human prostate cancer cells were obtained from the American Type Culture Collection (ATCC) and maintained in Roswell Park Memorial Institute (RPM) 1640 media supplemented with 10% fetal bovine serum and penicillin-streptomycin. All cells were maintained in a humidified incubator at 37° C. with 5% CO₂. 1000 cells per well were seeded into 96 well plates and allowed to incubate overnight.

The test compound was added in a matrix format in which COMPOUND A was added down the plate in an 8-point, 3-fold dilution starting at 5 μM to 2.3 nM and enzalutamide was added across the plate in an 8-point, 3-fold dilution dose curve from 20 μM to 9.1 nM. Cells were incubated for 12 days at 37° C. with 5% CO₂. CyQuant Direct Proliferation reagent (Invitrogen) was added per manufacturer's instructions and fluorescence was read on a Celigo cell counter. Data was analyzed with Chalice Bioinformatics Software v1.6 and ‘Synergy Score’ calculations generated where S=fcov ln fX ln fY Σ max(0,Idata) max(0,Idata-(Loewe), which is a positive-gated, inhibition-weighted volume over Loewe additivity. fX,Y are the dilution factors used for each single agent and the coverage factor fcov accounts for missing data, scaling the score up by the ratio of total/tested combination dose matrix points (https://horizondiscovery.com/-/media/Files/Horizon/resources/Technical-manuals/hd-technical-manual-chalice-analyzer-viewer.pdf).

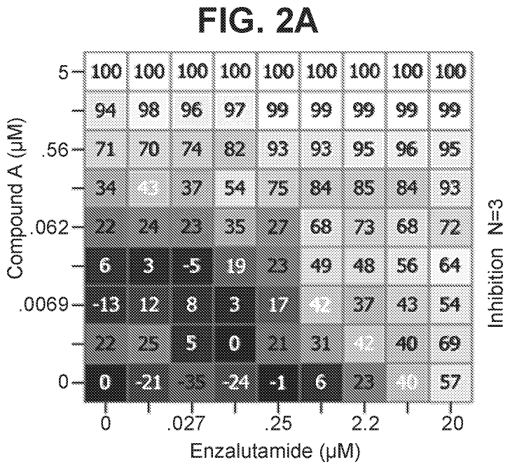

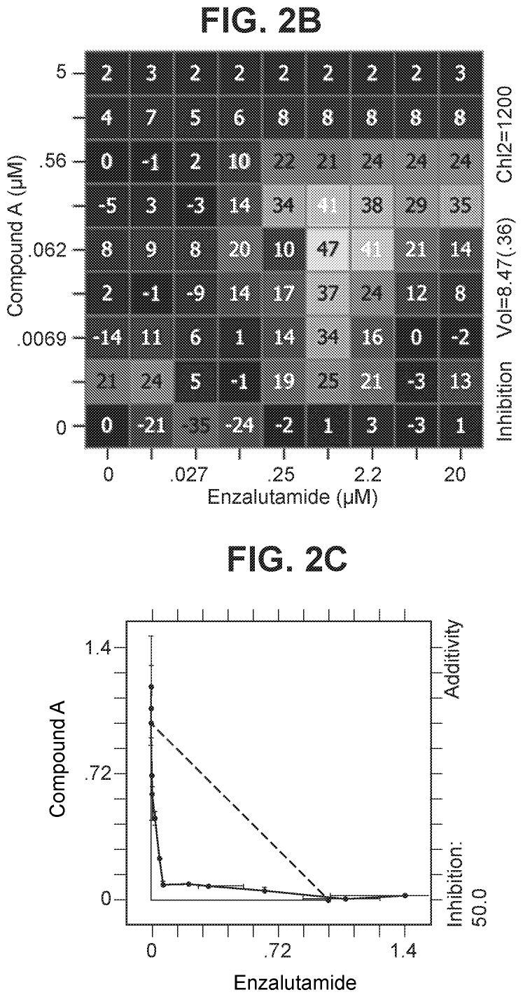

FIG. 2 shows a dose response matrix (A), Loewe excess matrix (B), and isobologram (C) demonstrating the effects of combining COMPOUND A and enzalutamide on proliferation of C4-3 cells over 12 days. FIG. 2A provides the full dose response matrix as a heat map showing compound activity, where darker colors and lower numbers (bottom left) indicate no or limited activity and lighter colors and higher numbers (upper right) indicate strong activity; a synergy score of 6.75 was calculated. FIG. 2B provides the Loewe excess matrix, which demonstrates synergy between Compound A and enzalutamide; a volume of 8.47 was calculated. FIG. 2C provides an isobologram depicting the dose combinations at which experimental inhibition (curve) exceeded additivity (diagonal).

### Example 3—In Vitro Screen in VCaP Human Prostate Cancer Cells

VCaP human prostate cancer cells were obtained from the American Type Culture Collection (ATCC) and maintained in Dulbecco's Modified Eagle's Medium (DMEM) media supplemented with Hyclone 10% fetal bovine serum (Non-HI), 1× Glutamax, and penicillin-streptomycin. All cells were maintained in a humidified incubator at 37° C. with 5% CO₂. 5000 cells per well were seeded into 96 well plates and allowed to incubate overnight.

The test compound was added in a matrix format in which COMPOUND A was added down the plate in an 8-point, 3-fold dilution starting at 5 μM to 2.3 nM and enzalutamide was added across the plate in an 8-point, 3-fold dilution dose curve from 20 μM to 9.1 nM. Cells were incubated for 15 days at 37° C. with 5% CO₂. CyQuant Direct Proliferation reagent (Invitrogen) was added per manufacturer's instructions and fluorescence was read on a Tecan M1000 plate reader. Data was analyzed with Chalice Bioinformatics Software v1.6 and ‘Synergy Score’ calculations generated where S=fcov ln fX ln fY Σ max(0,Idata) max(0,Idata-(Loewe), which is a positive-gated, inhibition-weighted volume over Loewe additivity. fX,Y are the dilution factors used for each single agent and the coverage factor fcov accounts for missing data, scaling the score up by the ratio of total/tested combination dose matrix points (https://horizondiscovery.com/-/media/Files/Horizon/resources/Technical-manuals/hd-technical-manual-chalice-analyzer-viewer.pdf).

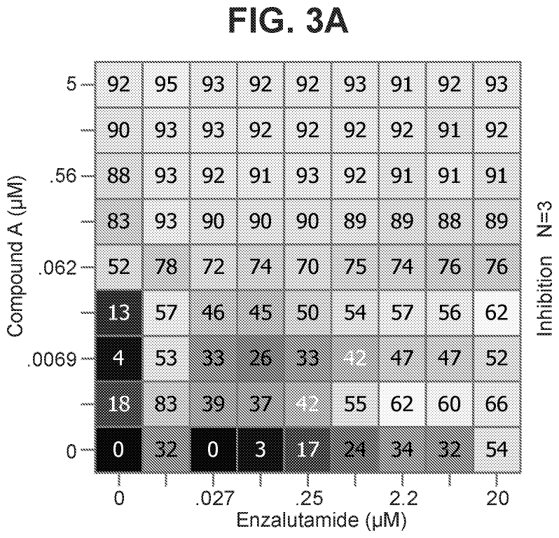

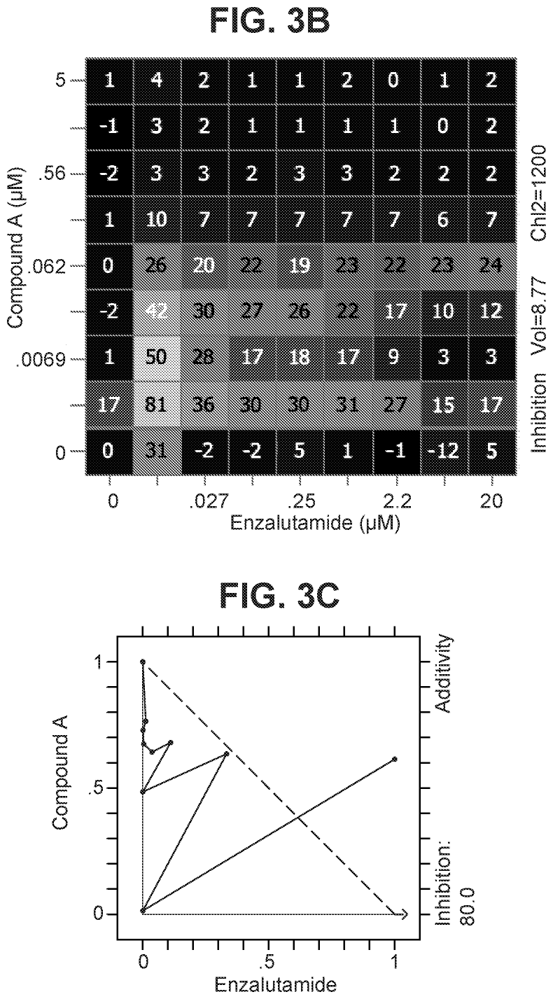

FIG. 3 shows a dose response matrix (A), Loewe excess matrix (B), and isobologram (C) demonstrating the effects of combining COMPOUND A and enzalutamide on proliferation of VCaP cells over 15 days. FIG. 3A provides the full dose response matrix as a heat map showing compound activity, where darker colors and lower numbers (bottom left) indicate no or limited activity and lighter colors and higher numbers (upper right) indicate strong activity; a synergy score of 6.55 was calculated. FIG. 3B provides the Loewe excess matrix, which demonstrates synergy between Compound A and enzalutamide; a volume of 8.77 was calculated. FIG. 3C provides an isobologram depicting the dose combinations at which experimental inhibition (curve) exceeded additivity (diagonal).

All publications and patent applications cited in the specification are herein incorporated by reference in their entirety. Although the foregoing invention has been described in some detail by way of illustration and example, it will be readily apparent to those of ordinary skill in the art in light of the teachings of this invention that certain changes and modifications may be made thereto without departing from the spirit or scope of the appended claims.

1. A method of treating cancer in a subject in need thereof comprising administering to the subject:
(a) an amount of a compound of Formula (I): 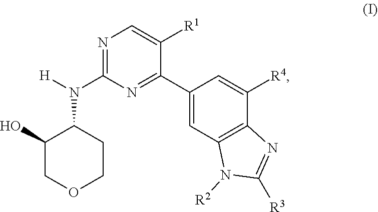
or a pharmaceutically acceptable salt thereof, wherein:
R¹ is H, For Cl;
R² is C₁-C₄ alkyl, where said C₁-C₄ alkyl is optionally substituted by R⁵;
R³ is H or C₁-C₄ alkyl, where said C₁-C₄ alkyl is optionally substituted by R⁶;
R⁴ is H or F; and
each R⁵ and R⁶ is independently OH, F or C₁-C₂ alkoxy; and
(b) an amount of an antiandrogen;
wherein the amounts in (a) and (b) together are effective in treating cancer.

2. The method of claim 1, wherein the compound of Formula (I) is 1,5-anhydro-3-({5-chloro-4-[4-fluoro-2-(2-hydroxypropan-2-yl)-1-(propan-2-yl)-1H-benz-imidazol-6-yl]pyrimidin-2-yl}amino)-2,3-dideoxy-D-threo-pentitol, or a pharmaceutically acceptable salt thereof.

3. The method of claim 1, wherein the antiandrogen is selected from the group consisting of enzalutamide, N-desmethyl enzalutamide, darolutamide, apalutamide, and abiraterone, or a pharmaceutically acceptable salt or solvate thereof.

4. The method of claim 3, wherein the antiandrogen is enzalutamide or a pharmaceutically acceptable salt or solvate thereof.

5. The method of claim 1, wherein the cancer is selected from the group consisting of prostate cancer, breast cancer, lung cancer, liver cancer, kidney cancer, bladder cancer, ovarian cancer, peritoneal cancer, fallopian tube cancer, cervical cancer, uterine cancer, pancreatic cancer, stomach cancer, colorectal cancer, esophageal cancer, head and neck cancer, testicular cancer, adrenal cancer, skin cancer, brain cancer, sarcoma, and lymphoma.

6. The method of claim 5, wherein the cancer is prostate cancer.

7. The method of claim 6, wherein the prostate cancer is metastatic prostate cancer (mPC).

8. The method of claim 6, wherein the prostate cancer is non-metastatic prostate cancer (nmPC).

9. The method of claim 6, wherein the prostate cancer is resistant to enzalutamide or abiraterone.

10. The method of claim 1, further comprising administering to the subject: (c) an amount of an additional anti cancer agent; wherein the amounts in (a), (b) and (c) together are effective in treating cancer.

11. The method of claim 10, wherein the additional anti-cancer agent is selected from the group consisting of an anti-tumor agent, an anti-angiogenesis agent, a signal transduction inhibitor, an antiproliferative agent, and an androgen deprivation therapy (ADT).

12. The method of claim 11, wherein the additional anti-cancer agent is an ADT.

13. The method of claim 12, wherein the ADT is selected from the group consisting of a gonadotropin releasing hormone (GnRH) agonist and a gonadotropin releasing hormone (GnRH) antagonist.

14. The method of claim 12, wherein the ADT is selected from the group consisting of leuprolide, buserelin, gonadorelin, goserelin, histrelin, nafarelin, triptorelin, deslorelin, fertirelin, abarelix, cetrorelix, degarelix, ganirelix, ozarelix, elagolix, relugolix and linzagolix, or a pharmaceutically acceptable salt thereof.

15. The method of claim 1, wherein the cancer is androgen dependent or androgen receptor (AR)-positive.

16-20. (canceled)

21. The method of claim 1, wherein the subject is human.

22. A method of treating cancer in a subject in need thereof comprising administering to the subject: (a) 1,5-anhydro-3-({5-chloro-4-[4-fluoro-2-(2-hydroxypropan-2-yl)-1-(propan-2-yl)-1H-benz-imidazol-6-yl]pyrimidin-2-yl}amino)-2,3-dideoxy-D-threo-pentitol, or a pharmaceutically acceptable salt thereof; and (b) enzalutamide or a pharmaceutically acceptable salt or solvate thereof; wherein the amounts in (a) and (b) together are effective in treating cancer.

23. The method of claim 22, wherein the cancer is prostate cancer.

24. The method of claim 23, wherein the prostate cancer is metastatic prostate cancer (mPC).

25. The method of claim 23, wherein the prostate cancer is non-metastatic prostate cancer (nmPC).
